(19) United States
(12) Patent Application Publication
(10) Pub. No.: US 2014/0377740 A1
(43) Pub. Date: Dec. 25, 2014

(54) SOLUBLE AND MEMBRANE-ANCHORED FORMS OF LASSA VIRUS SUBUNIT PROTEINS

(71) Applicant: The Administrators of the Tulane Educational Fund, New Orleans, LA (US)

(72) Inventors: Luis M. BRANCO, Germantown, MD (US); Alexander MATSCHINER, Rockville, MD (US); Megan M. ILLICK, Gaithersburg, MD (US); Darryl B. SAMPEY, Frederick, MD (US); Robert F. GARRY, New Orleans, LA (US); Daniel G. BAUSCH, New Orleans, LA (US); Joseph N. FAIR, Knoxville, MD (US); Mary C. GUTTIERI, Frederick, MD (US); Kathleen A. CASHMAN, Purcellville, VA (US); Russell B. WILSON, Mandeville, LA (US); Peter C. KULAKOSKY, Metairie, LA (US); F. Jon GESKE, Commerce City, CO (US)

(73) Assignees: BIOFACTURA, INC., Rockville, MD (US); THE ADMINISTRATORS OF THE TULANE EDUCATIONAL FUND, New Orleans, LA (US)

(21) Appl. No.: 14/168,438

(22) Filed: Jan. 30, 2014

Related U.S. Application Data
(63) Continuation of application No. 12/450,756, filed on Jun. 14, 2010, now abandoned.
(60) Provisional application No. 60/922,732, filed on Apr. 10, 2007.

(51) Int. Cl. C07K 14/005; G01N 33/569; C07K 16/10

(52) U.S. Cl. CPC C07K 14/005; C07K 16/10; G01N 33/56983; C07K 2319/30; C12N 2760/10022; C12N 2760/10033
USPC 435/5; 530/395; 530/387.3

(57) ABSTRACT

Soluble and membrane-anchored forms of Lassa virus (LASV) glycoprotein 1 (GP1), glycoprotein 2 (GP2), the glycoprotein precursor (GPC), the nucleocapsid protein (NP), and the nucleic acids encoding these proteins are disclosed, as well as diagnostic and preventative methods using these compositions. Also disclosed are methods including preparation of vaccines, factors (e.g. small molecules) that inhibit LASV infectivity, and diagnostic and therapeutic antibodies including neutralizing antibodies for the prevention and treatment of infection by LASV and other arenaviruses.

This application is a continuation of U.S. application Ser. No. 12/450,756, filed on Jun. 14, 2010, which is the National Phase of International Application No. PCT/US2008/004622, filed on Apr. 10, 2008, which claims the benefit of priority to U.S. Provisional Application No. 60/922,732, filed Apr. 10, 2007, each of which is incorporated herein by reference in its entirety.

### STATEMENT REGARDING FEDERALLY SPONSORED RESEARCH OR DEVELOPMENT

This invention was made, in part, with support provided by the United States government under Grant No. 1 UC 1 AI067188-01 awarded by the National Institute of Allergy and Infectious Diseases of the National Institutes of Health. The Government may have certain rights in this invention.

### FIELD OF THE INVENTION AND INCORPORATION OF SEQUENCE LISTING

This present invention relates to novel forms of protein subunits from Lassa virus (LASV), to compositions comprising the novel forms of protein subunits from LASV, and methods comprising the same. Biological sequence information for this application is included in an ASCII text file, filed with the application, having the file name “TU-360-CON1-SEQ.txt”, created on Jan. 28, 2014, and having a file size of 19,985 bytes, which is incorporated herein by reference.

### BACKGROUND

Lassa virus (LASV) and several other members of the Arenaviridae are classified as Biosafety Level 4 and NIAID Biodefense Category A agents. The proposed studies will fill a vital biodefense need for rapid multiagent immunodiagnostic assays for arenaviruses, and provide a major advance for public health management of an important family of viral pathogens.

Lassa Fever.

The most prevalent arenaviral disease is Lassa, an often-fatal hemorrhagic fever named for the Nigerian town in which the first described cases occurred in 1969 (Buckley and Casals, 1970). Parts of Guinea, Sierra Leone, Nigeria, and Liberia are endemic for the etiologic agent, LASV (Birmingham and Kenyon, 2001). Although detailed surveillance of LASV is hampered by many factors, including the lack of a widely available diagnostic test, it is clear that the public health impact is immense. There are as many as 300,000 cases of Lassa per year in West Africa and 5,000 deaths (see Centers for Disease Control website www(dot)cdc(dot)gov/ncidod/dvrd/spb/mnpages/dispages/lassaf(dot)htm). In some parts of Sierra Leone, 10-16% of all patients admitted to hospitals have Lassa fever. Case fatality rates for Lassa fever are typically 15% to 20%, although in epidemics overall mortality can be as high as 45%. LASV has been associated with severe nosocomial outbreaks involving health care workers and laboratory personnel (Fisher-Hoch et al., 1995). The mortality rate for women in the last month of pregnancy is always high, ˜90%, and LASV infection causes high rates of fetal death at all stages of gestation (Walls, 1985). Mortality rates for Lassa appear to be higher in non-Africans, which is of concern because Lassa is the most commonly exported hemorrhagic fever (Haas et al., 2003; Holmes et al., 1990).

Old and New World Arenaviruses.

The genome of arenaviruses consists of two segments of single-stranded, ambisense RNA. There are three major structural proteins, including two envelope glycoproteins (GP1 and GP2) and the nucleocapsid protein (NP). The structure of arenavirus GP2 appears to be a class I fusion protein, which is common to envelope glycoproteins of myxoviruses, retroviruses and filoviruses (Gallaher, DiSimone, and Buchmeier, 2001). When viewed by transmission electron microscopy, the enveloped spherical virions (diameter: 110-130 nm) show grainy particles that are ribosomes acquired from the host cells (Murphy and Whitfield, 1975). Hence the use for the family name of the Latin word “arena,” which means “sandy.” The arenaviruses are divided into the Old World or lymphocytic choriomeningitis virus (LCMV)/LASV complex and the New World or Tacaribe complex (Bowen, Peters, and Nichol, 1997). There is considerable diversity amongst members of the Arenaviridae (FIG. 1), and even within the same virus species (Bowen et al., 2000). In addition to LASV, other arenaviruses that cause severe illness in humans and are classified as BSL-4 and NIAID category A agents, include the New World arenaviruses Machupo virus (MACV, Bolivian hemorrhagic fever), Junin virus (JUNV, Argentine hemorrhagic fever), Guanarito virus (GUAV, Venezuelan hemorrhagic fever) and Sabiá virus (SABV, Brazilian hemorrhagic fever). Arenaviruses are zoonotic; each virus is associated with a specific species of rodent (Bowen, Peters, and Nichol, 1997). The LCMV/LASV complex viruses are associated with Old World rats and mice (family Muridae, subfamily Murinae). Tacaribe complex viruses are generally associated with New World rats and mice (family Muridae, subfamily Sigmodontinae); however, the reservoir of Tacaribe virus itself appears to be a bat (Bowen, Peters, and Nichol, 1996). The reservoir of LASV is the “multimammate rat” of the genus Mastomys (Monath et al., 1974). Mastomys rats are ubiquitous in sub-Saharan Africa (Demby et al., 2001) and are known to be peridomestic, often living in human homes; however, many questions regarding the taxonomy, geographic distribution and ecobiology of Mastomys species are unanswered. As with the natural hosts of other arenaviruses, Mastomys show no symptoms of LASV infection, but shed the virus in saliva, urine and feces. Eradication of the widely distributed rodent reservoirs of LASV and other arenaviruses is impractical and ecologically undesirable.

Arenaviruses are easily transmitted to humans via direct contact with rodent excreta or by contact with or ingestion of excreta-contaminated materials (Bausch et al., 2001; Demby et al., 2001). Infection usually occurs via mucous membranes or skin breaks. In the case of Mastomys species, infection may also occur when the animals are caught, prepared as a food source and eaten. Most arenaviruses, including LASV, are readily transmitted beTWEEN humans, thus making nosocomial infection another matter of great concern. Human-to-human transmission can occur via exposure to blood or body fluids. LASV can also be transmitted to sexual partners of convalescent men via semen up to six weeks post-infection.

Natural History of Lassa Fever.

Signs and symptoms of Lassa fever, which occur 1-3 weeks after virus exposure, are highly variable, but typically begin with the insidious onset of fever and other nonspecific symptoms such as headache, generalized weakness, and malaise, followed within days by sore throat, retrosternal pain, conjunctival injection, abdominal pain, and diarrhea. LASV infects endothelial cells, resulting in increased capillary permeability, which can produce diminished effective circulating volume (Peters et al., 1989). Severe cases progress to facial and neck swelling, shock and multiorgan system failure. Frank bleeding, usually mucosal (gums, etc.), occurs in less than a third of cases, but confers a poor prognosis. Neurological problems have also been described, including hearing loss, tremors, and encephalitis. Patients who survive begin to defervesce 2-3 weeks after onset of the disease. Temporary or permanent unilateral or bilateral deafness that occurs in a third of Lassa patients during convalescence is not associated with the severity of the acute disease (Cummins et al., 1990; Rybak, 1990).

Potential for Use of Arenaviruses as Bioweapons.

In addition to high case fatality rates, arenaviruses have many features that enhance their potential as bioweapons. Arenaviruses have relatively stable virions, do not require passage via insect vectors, are spread easily by human-to-human contact and may be capable of aerosol spread or other simple means of dispersal. The high prevalence of Lassa fever in western Africa coupled with the ease of travel to and from this area and endemic areas for MACV, JUNV, GUAV, SABV and other highly pathogenic arenaviruses permits easy access to these viruses for use as a bioweapon. A cluster of hemorrhagic fever cases in the United States caused by any arenavirus would be a major public health incident. Because febrile illnesses are common, the absence of reliable diagnostic tests would greatly increase the impact of the attack and permit wider dissemination via human-to-human contact. The potential use of LASV and other arenaviruses as a biological weapon directed against civilian or military targets necessitates the commercial development of effective diagnostics.

Treatment/Prevention of Arenavirus Infections.

The antiviral drug ribavirin is effective in the treatment of Lassa fever if administered early in the course of illness (Johnson et al., 1987; McCormick et al., 1986). Ribavirin administered to patients with a high virus load (and therefore a high risk for mortality) within the first six days of illness reduced the case-fatality rate from 55% to 5% (McCormick et al., 1986). Several anecdotal reports suggest that this drug can also be effective against other arenaviral hemorrhagic fevers (Barry et al., 1995; Kilgore et al., 1997; Weissenbacher et al., 1986a; Weissenbacher et al., 1986b). The efficacy of prophylactic treatments for Lassa fever is unknown, although it has been suggested that people with high-risk exposures be treated with oral ribavirin. Passive transfer of neutralizing antibodies early after infection may also be an effective treatment for Lassa and other arenaviral hemorrhagic fevers (Enria et al., 1984; Frame et al., 1984; Jahrling, 1983; Jahrling and Peters, 1984; Jahrling, Peters, and Stephen, 1984; Weissenbacher et al., 1986a). The dependence of effective treatment on early diagnosis provides another strong rationale for improving arenavirus diagnostics. No arenavirus vaccine is currently available, although vaccines against LASV and JUNV are in development. Effective diagnostic assays are absolutely essential for development and field testing arenaviral vaccines.

Diagnostic Procedures for Arenaviruses.

Virus isolation is likely to be the most sensitive assay for detection of LASV and other arenaviruses. However, LASV cannot be uniformly isolated from all acute cases (Bausch et al., 2000; Johnson et al., 1987). Virus culture is too time-consuming for bioterrorism scenarios or clinical settings given the urgency that effective treatment requires. The diversity amongst LASV isolates and the number of other arenaviruses that are potential bioterrorism agents suggests that it may be impractical to develop a useful RT-PCR strategy for rapid detection (Archer and Rico-Hesse, 2002; Bowen, Peters, and Nichol, 1997; Niedrig et al., 2004). The recent International Quality Assurance Study on the Rapid Detection of Viral Agents of Bioterrorism by RT-PCR methods recently found that only a fraction (21-50%) of established biodefense laboratories could detect common strains of LASV in samples containing fewer than 5000 copies/ml (Niedrig et al., 2004). Furthermore, PCR methods require instrumentation, expertise and facilities generally not available in LASV endemic areas of West Africa (Demby et al., 1994; Lunkenheimer, Hufert, and Schmitz, 1990; Trappier et al., 1993). Our prior results (Bausch et al. 2000) strongly suggest that antigen-capture and IgM-capture ELISA provide the most sensitive and specific serologic tests for acute Lassa virus infection as well as useful prognostic information. We anticipate that similar assays can be developed for New World arenaviruses, which also have high potential for use as bioterrorism agents. This application is based on the premise that, as was the case with advanced generation HIV antibody tests, arenavirus ELISA can be developed with superior sensitivity and specificity compared to currently available noncommercializable assays. Prior studies readily demonstrated the feasibility of this approach (Barber, Clegg, and Lloyd, 1990; Hufert, Ludke, and Schmitz, 1989; Jahrling, Niklasson, and McCormick, 1985; Krasko et al., 1990; Meulen et al., 2004; Vladyko et al., 1990). Additional advantages of ELISA-based diagnostics include their ease of standardization and use (in comparison to PCR-based assays), and their applicability to the diagnosis of numerous other diseases. It should be possible to combine LASV detection with detection for selected pathogens that have a clinical presentation similar to Lassa fever such as Ebola virus or dengue virus. ELISA can be converted to formats that would be especially valuable for rapid diagnosis during an incident of bioterrorism and could be used in technology-poor regions such as West Africa.

Need for the Invention.

The scientific literature describing the expression of LASV proteins in prokaryotic systems, such as E. coli, report only expression of polypeptide fragments. The expression of truncated forms of LASV nucleocapsid protein in E. coli BL21(DE3) has been reported by Jan ter Meulen et al. (1998 and 2000). In these reports Jan ter Meulen et al. stated that “Neither the whole NP nor the N terminus (amino acids [aa] 1 to 139) could be expressed (data not shown), but a truncated protein (aa 141 to 569) was abundantly overexpressed, extracted from insoluble inclusion bodies with 8 M urea, and purified by nickel-chelate chromatography”. The expression of full length LASV nucleocapsid protein in insect cells mediated by infection with a recombinant baculovirus encoding the entire open reading frame of this polypeptide has been reported by L. S. Lukashevich et al. (1993). Furthermore, at the time of this invention no reports in the literature have described expression of truncated fragments or full length LASV GP1 or GP2 polypeptides in prokaryotic systems.

Thus, development of broad encompassing and highly specific immunological-based diagnostic procedures that use recombinant LASV proteins requires successful expression, purification, and characterization of full length versions of NP, GP1, and GP2. Full length expression of each polypeptide, as outlined in this application, required a rationally designed and empirically optimized approach that utilized specific E. coli strains, fusion to a partner protein that both stabilized expression and could be used as a purification domain, expression of rare tRNA codons in the bacterial cell to support efficient translation of the recombinant LASV proteins, and a matrix-based identification of culture medium, induction, and temperature conditions. In addition, localization of recombinant LASV proteins to two independent cellular compartments was investigated. Furthermore, purification and stabilization procedures were developed for each of the LASV proteins individually, as each polypeptide required specific conditions for optimal purification and solubility in aqueous solutions. Optimal expression, purification, and solubilization formats were subsequently chosen for each LASV protein, and the processes were standardized. Specific examples are outlined in the body of this application.

### SUMMARY OF INVENTION

The present invention discloses compositions comprising soluble and membrane-anchored forms of Lassa virus (LASV) glycoprotein 1 (GP1), glycoprotein 2 (GP2), the glycoprotein precursor (GPC), and the nucleocapsid protein (NP). Another embodiment of the present invention is drawn to proteins that consist of soluble and membrane-anchored forms of Lassa virus (LASV) glycoprotein 1 (GP1), glycoprotein 2 (GP2), the glycoprotein precursor (GPC), and the nucleocapsid protein (NP). This invention also relates to diagnostic and preventative methods using the novel forms of the LASV subunit proteins. Preventative methods include preparation of vaccines, as well as factors (e.g. small molecules, peptides) that inhibit LASV infectivity. Further, the invention relates to diagnostic and therapeutic antibodies including neutralizing antibodies for the prevention and treatment of infection by LASV and other arenaviruses. The present invention also discloses and provides new tools and methods for the design, production, and use of soluble and membrane-anchored forms of LASV GP1, GP2, NP and GPC including expression in engineered bacterial- and mammalian-based systems.

One embodiment of the invention relates to polynucleotides and polypeptides or fragments thereof encoding soluble forms of LASV GP1. The polynucleotide sequences may encode polypeptides that comprise or consist of soluble forms of LASV GP1 or fragments thereof.

Another embodiment of the invention relates to polynucleotides and polypeptides or fragments thereof encoding soluble forms of LASV GP2. The polynucleotide sequences may encode polypeptides that comprise or consist of soluble forms of LASV GP2 or fragments thereof.

Another embodiment of the invention relates to polynucleotides and polypeptides or fragments thereof encoding membrane-anchored forms of LASV GPC. The polynucleotide sequences may encode polypeptides that comprise or consist of membrane-anchored forms of LASV GPC or fragments thereof.

Another embodiment of the invention relates to polynucleotides and polypeptides or fragments thereof encoding a form of LASV NP. The polynucleotide sequences may encode polypeptides that comprise or consist of LASV NP or fragments thereof.

Another embodiment of the invention relates to methods of producing forms of LASV GP1, GP2, GPC, and NP.

Another embodiment of the invention relates to expression vectors comprising polynucleotides encoding forms of LASV GP1, GP2, GPC, and NP.

Another embodiment of the invention relates to fusion proteins comprising a polypeptide of the invention and one or more polypeptides that enhance the stability of a polypeptide of the invention and/or assist in the purification of a polypeptide of the invention.

An embodiment of the invention relates to antibodies or fragments thereof, such as neutralizing antibodies, specific for one or more polypeptides of the invention and diagnostic and/or therapeutic application of such antibodies.

Another embodiment of the invention relates to diagnostics comprising the polypeptides of the invention and/or antibodies or fragments thereof including labeled antibodies or fragments thereof of the invention.

Another embodiment of the invention relates to a subunit vaccine comprising the polynucleotides or polypeptides of the invention.

Another embodiment of the invention is directed to kits comprising the polynucleotides, polypeptides, and/or antibodies of the invention.

Other embodiments and advantages of the invention are set forth in part in the description, which follows, and in part, may be obvious from this description, or may be learned from the practice of the invention.

### DESCRIPTION OF THE TABLES

Table 1 describes the oligonucleotide primers used for amplification of LASV genes for expression in E. coli.

Table 2 describes the oligonucleotide primers used for amplification of LASV genes for expression in mammalian cells.

Table 3 is a summary of vectors and respective E. coli strains used to express recombinant LASV genes.

Table 4 is a summary of vectors and respective mammalian cell lines used to express recombinant LASV genes.

Table 5 summarizes studies for invention production phase 1 as described in Example 10.

Table 6 summarizes studies for invention production phase 2 as described in Example 10.

Table 7 presents data showing that the recombinant IgM capture ELISA is a much faster assay (approximately 1.5 hours) than the traditional IgM capture assay, which takes over 6 hours (refer to Example 10).

Table 8 presents comparisons of recombinant LASV ELISA with traditional ELISA and PCR detection using a serological panel from the Kenema Government Hospital Lassa Ward (refer to Example 10).

Table 9 shows IgM and IgG reactivity to recombinant LASV proteins in a cohort of follow-up patients from the Lassa Ward of Kenema Government Hospital and their household contacts (refer to Example 10).

### DESCRIPTION OF THE FIGURES

FIG. 1 depicts the phylogenetic relationships among the members of the family Arenaviridae. Partial NP gene nucleotide sequences were aligned and analyzed by maximum parsimony (redrawn from Bowen, Peters, and Nichol 1996. See also Bowen et al., 2000).

FIG. 2 depicts the cloning strategy for expression of LASV proteins (A) GP1, GP2, and NP in E. coli using pMAL vectors and (B) GPC, GP1, and GP2 in mammalian cells using the human cytomegalovirus (CMV) promoter-driven eukaryotic vectors. (A) To generate MBP-LASV gene fusions for E. coli expression, PCR-amplified LASV gene sequences were restricted and cloned in-frame at the 3′ end of the malE gene, beyond the cleavage site for Factor Xa (IQGR). The LASV GP1 gene sequence comprised amino acids (a.a.) 59-259 in the native GPC, spanning the first a.a. beyond the known signal peptidase (SPase) cleavage site at position 58 to the junction beTWEEN GP1 and GP2 domains, which is cleaved by the SK1 protease at a.a. 259. The LASV GP2 gene sequence comprised a.a. 260-427, spanning the first a.a. of mature GP2 to the last a.a. before the predicted transmembrane (TM) domain. The LASV NP gene sequence comprised the complete ORF of the gene, with the exception of the N-terminal methionine. The 3′ oligonucleotides used for amplification of each gene sequence were engineered to contain two termination codons separated by a single nucleotide. All genes were cloned into vectors pMAL-p2X and pMAL-c2X for periplasmic and cytoplasmic expression of fusion proteins, respectively, in E. coli Rosetta 2(DE3)] or -gami 2 strains. The a.a. position of each LASV gene domain is noted. Abbreviations include: maltose binding protein (MBP), MBP gene (malE), MBP promoter (Ptac), transmembrane (TM), filamentous phage origin of replication (M13 ori), bacterial origin of replication (pBR322 ori), beta-lactamase gene (bla), E. coli transcription terminator (rrnB), the LacZ alpha-complementation domain (LacZα), and the lad repressor gene (lacIq). The periplasmic secretory domain in pMAL-p2x is indicated by a small box on the 5′ end of the malE gene sequence. The multiple cloning site sequence shown for the pMAL vectors is SEQ ID NO:1. The Ile-Glu-Gly-Arg amino acid sequence shown before the factor Xa cleavage site is SEQ ID NO:2. (B) For LASV protein expression in transiently transfected or stably transfected mammalian cell lines, the following were cloned into a pcDNA3.1/Zeo(+) vector background: (i) the complete GPC coding sequence (also termed pre-GPC for its inclusion of a signal sequence); (ii) the ectodomain of GP1, containing the native GPC signal peptide (SP) and fused to the GP2 TM domain on the C-terminus of the protein; and the ectodomain of GP2 fused to (iii) a human IgG lambda light chain (h λ LC) or (iv) human heavy chain (h HC) signal peptide sequence and retaining the native TM domain. Genes were cloned either in a vector lacking (pcDNA3.1/Zeo[+]) or containing (pcDNA3.1/Zeo[+]:intA) the CMV intron-A sequence. To generate soluble GP1 and GP2, the following were cloned into a pBFdhfr vector background: the ectodomain of GP1, containing the native GPC SP, and (v) a C-terminal FLAG-tag or (vi) no FLAG-tag (vi); the ectodomain of GP1 fused to (vii) a h λ LC or (viii) a h HC signal sequence; the ectodomain of GP2 fused to (ix) a h λ LC or (x) a h HC signal sequence; and the ectodomain of GP2 fused to (xi) a h HC signal sequence and a C-terminal FLAG-tag. Genes were cloned in a vector either lacking (pBFdhfr.1) or containing (pBFdhfr.2) the CMV intron-A sequence. Predicted N-linked glycosylation sites on GPC are indicated by “Y” symbols. The a.a. position of each LASV gene domain is noted. Values in parenthesis represent domain positions relative to the native GPC sequence. Abbreviations include: signal peptide (SP), amino terminus (NH2), carboxyl terminus (COOH), transmembrane (TM), bacterial origin of replication (ori), beta-lactamase gene (bla), Cytomegalovirus early promoter (PCMV), Bovine Growth Hormone polyadenylation signal (BGHpA), single stranded philamentous phage origin (f1), Simian Virus 40 origin of replication (SV40 ori), Simian Virus 40 polyadenylation signal (SV40 pA), dihydrofolate reductase gene (dhfr), and purification tag sequence DYKDDDDK (FLAG) (SEQ ID NO:3).

FIG. 3 depicts the Lassa virus (LASV) nucleocapsid protein (NP) nucleotide (SEQ ID NO:4) and amino acid (SEQ ID NO:5) sequences, the Lassa virus pre-glycoprotein precursor protein (Pre-GPC) nucleotide (SEQ ID NO:6) and amino acid (SEQ ID NO:7) sequences, a human IgG lambda light chain signal sequence nucleotide (SEQ ID NO:8) and amino acid (SEQ ID NO:9) sequences, and a human IgG heavy chain signal sequence nucleotide (SEQ ID NO:10) and amino acid (SEQ ID NO:11) sequences.

FIG. 4 illustrates laboratory analysis of patients admitted to the Lassa Fever Ward of the Kenema Government Hospital from October 2006-September 2007. Patients were considered confirmed if found positive by antigen-capture ELISA. Patients were considered probable if they were found to be positive for Lassa virus-specific IgM antibodies, but no antigen. The numbers of deaths are those associated with confirmed Lassa virus cases (note: the serological panel includes many of these patients, plus several additional patient samples). Updated numbers in September include an additional four patients.

FIG. 5: Antigen levels in serum from patient G038 over time. Absolute OD readings are shown (no background is subtracted). Gamma-irradiated slurries from LASV-infected and mock-infected cells are used as positive and negative controls, respectively. N/C is a normal control serum. Antigen levels were measured in patient G038 over 1-8 days. Samples G038-1 and -3 were PCR-positive. None of the samples were positive by the traditional IgM (tIgM) capture ELISA, but samples after G038-2 were positive by GP1 and GP2 recombinant IgM capture.

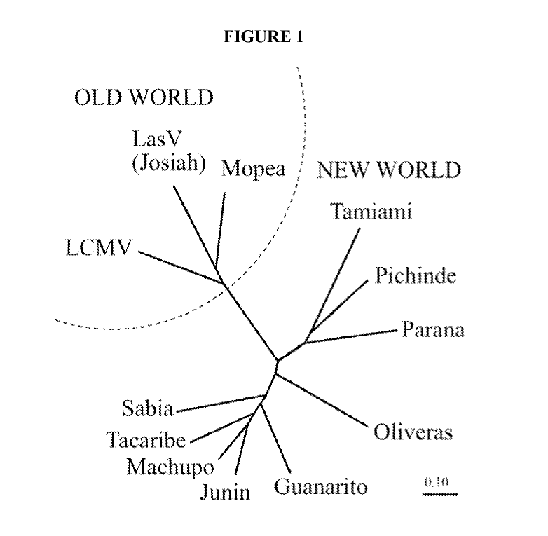

### DETAILED DESCRIPTION OF THE INVENTION

### General Techniques

The practice of the present invention will employ, unless otherwise indicated, conventional techniques of molecular biology (including recombinant techniques), microbiology, cell biology, biochemistry, and immunology, which are all within the normal skill of the art. Such techniques are fully explained in the literature, such as, for example, Molecular Cloning: A Laboratory Manual, second edition (Sambrook, et al., 1989) Cold Spring Harbor Press; Methods in Molecular Biology, Humana Press; Cell Biology: A Laboratory Notebook (I. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cabs, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

As used herein, the singular form “a”, “an”, and “the” includes plural references unless indicated otherwise. For example, “a” soluble glycoprotein includes one or more soluble glycoproteins.

Generally, this invention provides soluble and membrane-anchored forms of LASV protein subunits, the polynucleotides encoding the proteins, and methods for using these proteins in diagnosis, detection, and treatment. Specifically, this invention provides soluble forms of NP, soluble and membrane-anchored forms of LASV GP1 and GP2, and membrane-anchored forms of GPC protein subunits which retain characteristics of the native viral protein subunits allowing for development and production of diagnostics, vaccines, therapeutics, and screening tools.

Generally, the soluble forms of LASV GP1 and GP2 comprise all or part of the ectodomains of the native LASV GP1 and GP2 protein subunits. Soluble forms of GP1 and GP2 are generally produced by expressing GP1 and GP2 separately and deleting all or part of the transmembrane domain (TM) of the native mature LASV GP2 subunit protein and deleting all or part of the intracellular c-terminus domain (IC) of the native mature LASV GP2 subunit protein. By way of example, a soluble LASV GP2 glycoprotein may comprise the complete ectodomain of the native mature LASV GP2 glycoprotein.

The term ectodomain refers to that portion of a protein which is located on the outer surface of a cell. For example, the ectodomain of a transmembrane protein is that portion(s) of the protein which extends from a cell's outer surface into the extracellular space (e.g. the extracellular domain of the mature native LASV GP2; refer to amino acids 260-427 of the GPC). Further, an ectodomain can describe entire proteins that lack a transmembrane domain, but are located on the outer surface of a cell (e.g. mature native LASV GP1; refer to amino acids 59-259 of the GPC).

Generally, the soluble forms of LASV NP comprise all or part of the primary amino acid sequence of the native LASV NP protein subunit.

Generally, the membrane-anchored forms of LASV GP1, GP2, and GPC comprise, respectively, all or part of the ectodomains of the native LASV GP1, GP2, and GPC protein subunits fused to a TM and/or other sequences. By way of example and not limitation, a membrane-anchored LASV GP2 glycoprotein may comprise the complete ectodomain of the native mature LASV GP2 glycoprotein, the complete TM of the native mature LASV GP2 glycoprotein, the complete IC of the native mature LASV GP2 glycoprotein, and the secretory peptide (SP) sequence of a human IgG λ light chain. Also by way of example and not limitation, a membrane-anchored LASV GP1 glycoprotein may comprise the complete ectodomain of the native mature LASV GP1 glycoprotein, a sequence identical to the complete TM of the native mature LASV GP2 glycoprotein including an additional three amino acids from the predicted GP2-IC, and the SP sequence of the LASV GPC glycoprotein precursor.

The novel forms of LASV GP1, GP2, GPC, and NP of the invention generally retain one or more of the characteristics of the native viral protein subunits such as the ability to elicit antibodies (including, but not limited to, viral neutralizing antibodies) or the ability to interact or bind antibodies found in serum of animals (including humans) that have been exposed to LASV (i.e., Lassa Fever convalescent patient sera). Conventional methodology may be utilized to evaluate the novel forms of LASV GP1, GP2, GPC, and NP of the invention for one or more of these characteristics. Examples of such methodology that may be used include, but are not limited to, the assays described herein in the Examples.

### Polynucleotides

The term polynucleotide is used broadly and refers to polymeric nucleotides of any length (e.g., oligonucleotides, genes, small inhibiting RNA, fragments of polynucleotides encoding a protein, etc). By way of example and not limitation, the polynucleotides of the invention may comprise a sequence encoding all or part of the ectodomain and part of the transmembrane domain. The polynucleotide of the invention may be, for example, linear, circular, supercoiled, single-stranded, double-stranded, branched, partially double-stranded or partially single-stranded. The nucleotides comprised within the polynucleotide may be naturally occurring nucleotides or modified nucleotides. Generally the polynucleotides of the invention encode for all or part of the ectodomain (i.e. extracellular domain) of LASV GP1, GP2, and GPC; the full LASV NP; and all or part of the ectodomains of the native LASV GP1, GP2, and GPC protein subunits fused to a TM and/or other sequences.

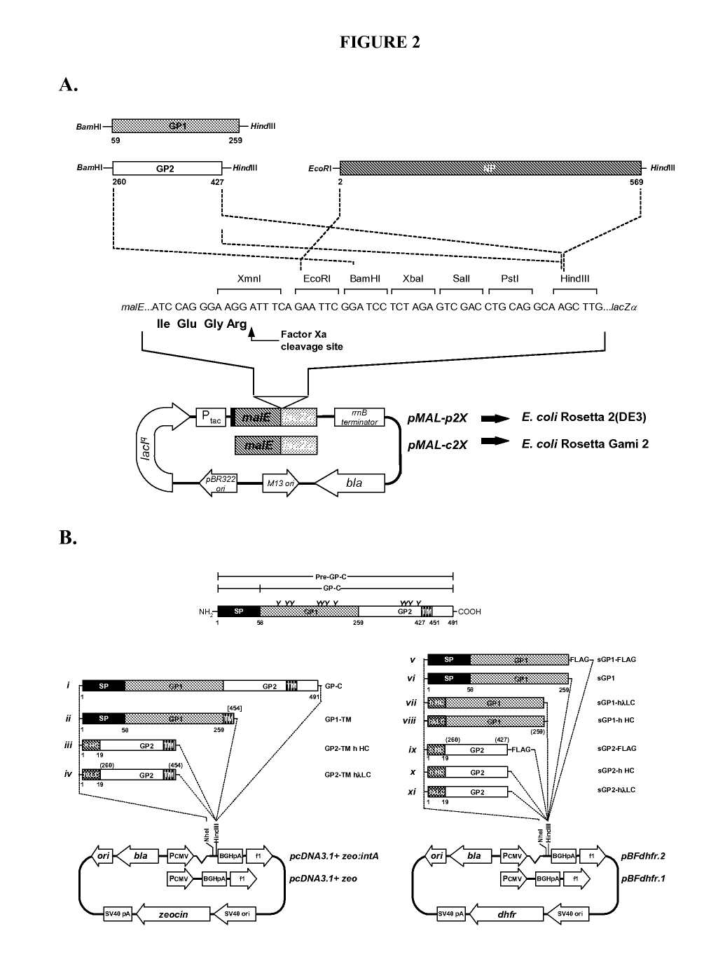

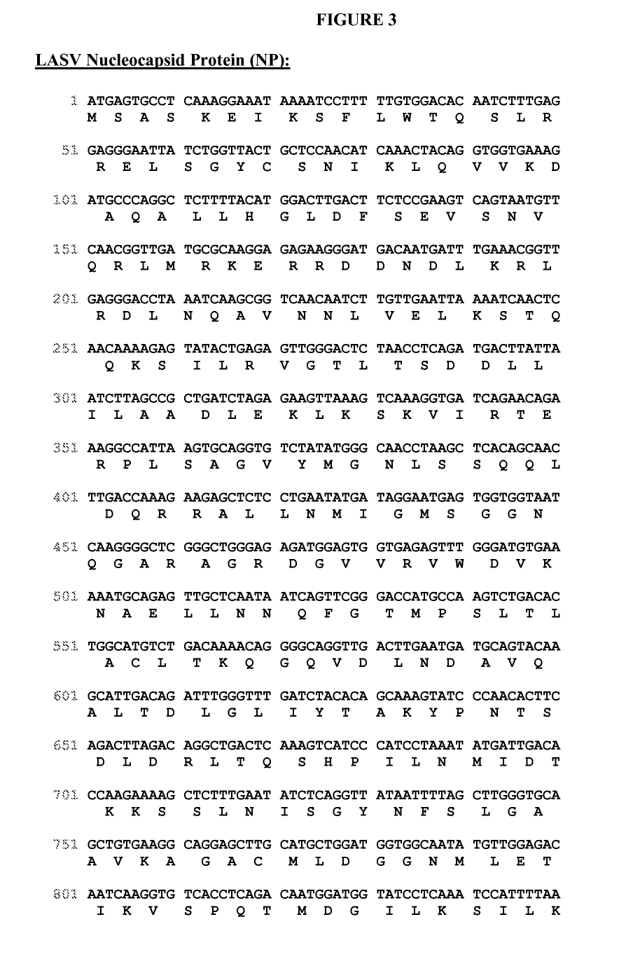

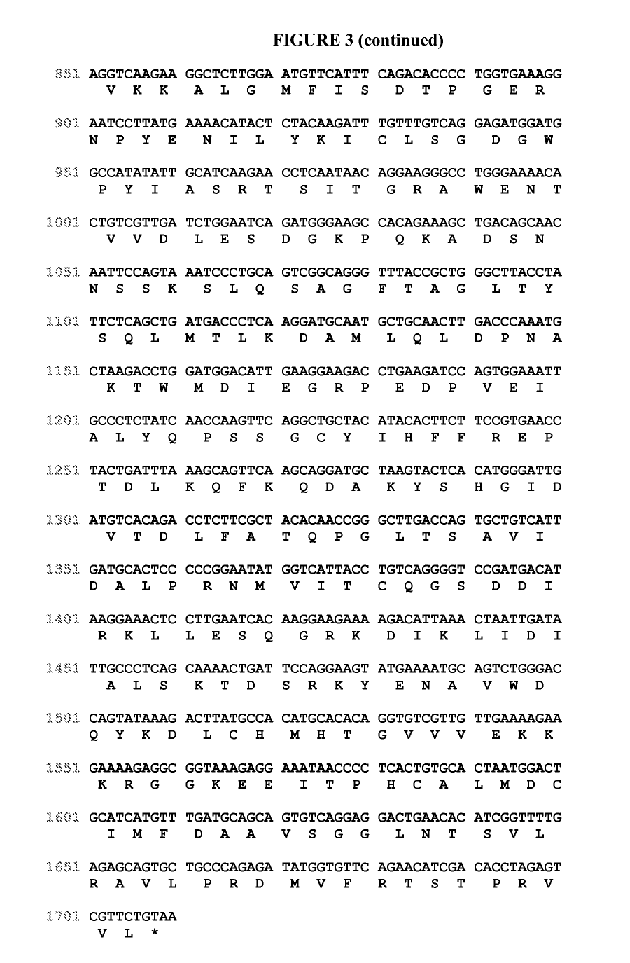

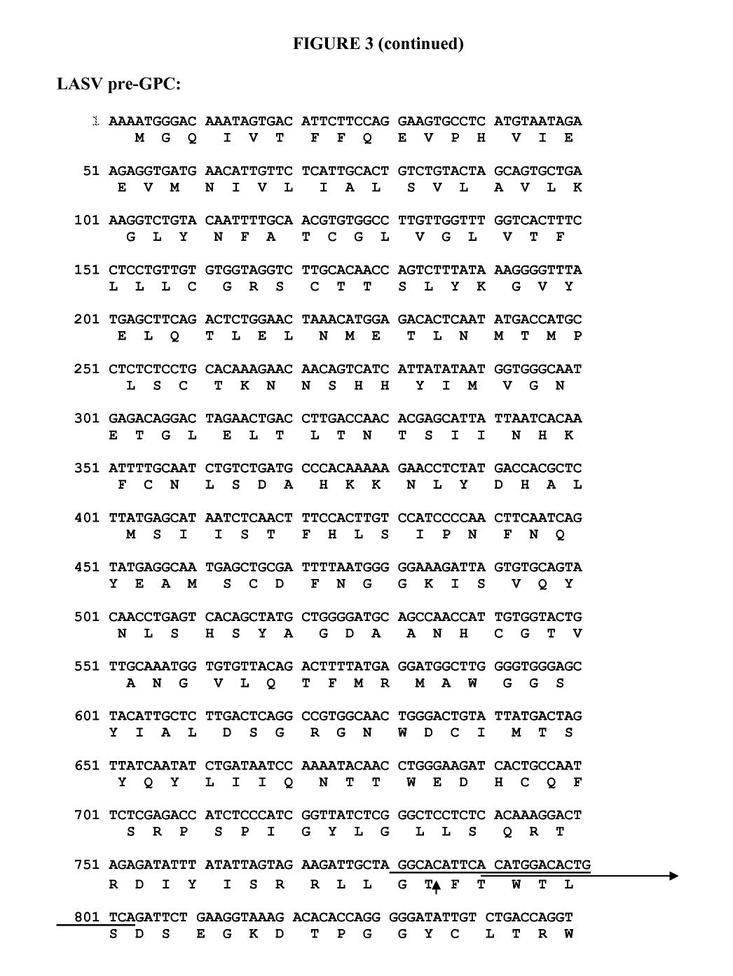

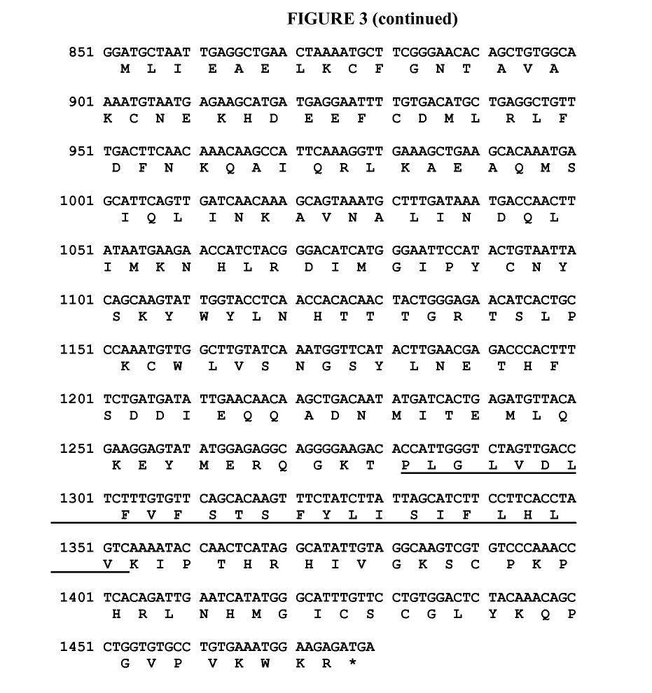

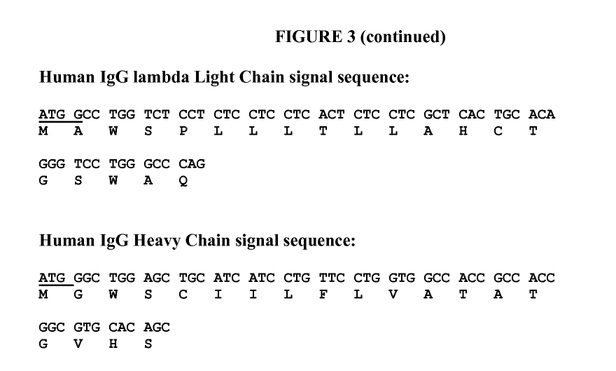

Generally, glycoprotein and nucleocapsid protein sequences from any Lassa virus isolate or strain may be utilized to derive the polynucleotides and polypeptides of the invention. By way of example and not limitation, a polynucleotide encoding a soluble LASV GP2 glycoprotein may comprise a polynucleotide sequence encoding amino acid residues 260-427 of the glycoprotein precursor protein (GPC, FIG. 2B), which residues represent the GP2 protein lacking its transmembrane and intracellular domains, linked to a polynucleotide encoding a human IgG λ light chain or a human IgG heavy chain signal sequence fused to the N-terminus of GP2. Also by way of example and not limitation, a polynucleotide encoding a soluble LASV GP1 glycoprotein may comprise a polynucleotide sequence encoding amino acid residues 1-58 of the LASV GPC, which residues represent the signal sequence, and a polynucleotide sequence encoding amino acid residues 59-259 of the GPC, which residues represent the mature LASV GP1 protein. Also by way of example and not limitation, a polynucleotide encoding a membrane-anchored LASV GP2 glycoprotein may comprise a polynucleotide sequence encoding amino acid residues 260-451 of the LASV GPC, which residues represent the mature LASV GP2 protein with its transmembrane domain, with a polynucleotide sequence encoding a human IgG λ light chain or a human IgG heavy chain signal sequence fused to the N-terminus of the GP2 protein.

Functional equivalents of these polynucleotides are also intended to be encompassed by this invention. By way of example and not limitation, functionally equivalent polynucleotides are those that encode a soluble glycoprotein of LASV and possess one or more of the following characteristics: the ability to elicit antibodies (including, but not limited to, viral neutralizing antibodies) capable of recognizing native LASV polypeptides or the ability to interact with or bind antibodies found in serum of animals (including humans) that have been exposed to LASV (i.e., Lassa Fever convalescent patient sera). Functional polynucleotide equivalents include those sequences that vary by virtue of the degenerate nature of the DNA code (i.e. different codons may encode the same amino acid). This degeneracy permits the expression of the same protein from different polynucleotide sequences.

Polynucleotide sequences which are functionally equivalent may also be identified by methods known in the art. A variety of sequence alignment software programs are available to facilitate determination of homology or equivalence. Non-limiting examples of these programs are BLAST family programs including BLASTN, BLASTP, BLASTX, TBLASTN, and TBLASTX (BLAST is available from the worldwide web at ncbi(dot)nlm(dot)nih(dot)gov/BLAST/), FASTA, COMPARE, DOTPLOT, BESTFIT, GAP, FRAMEALIGN, CLUSTALW, and PILEUP. Other similar analysis and alignment programs can be purchased from various providers such as DNA Star's MEGALIGN, or the alignment programs in GENEJOCKEY. Alternatively, sequence analysis and alignment programs can be accessed through the world wide web at sites such as the CMS Molecular Biology Resource at sdsc(dot)edu/ResTools/cmshp(dot)html and EXPASY Proteomics Server at www(dot)expasy(dot)ch/. Any sequence database that contains DNA or protein sequences corresponding to a gene or a segment thereof can be used for sequence analysis. Commonly employed databases include but are not limited to GENBANK, EMBL, DDBJ, PDB, SWISS-PROT, EST, STS, GSS, and HTGS.

Parameters for determining the extent of homology set forth by one or more of the aforementioned alignment programs are well established in the art. They include but are not limited to p value, percent sequence identity and the percent sequence similarity. P value is the probability that the alignment is produced by chance. For a single alignment, the p value can be calculated according to Karlin et al. (1990) Proc. Natl. Acad. Sci. (USA) 87: 2246. For multiple alignments, the p value can be calculated using a heuristic approach such as the one programmed in BLAST. Percent sequence identify is defined by the ratio of the number of nucleotide or amino acid matches beTWEEN the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical.

### Polypeptides

Another aspect of this invention is directed to soluble LASV GP1 and GP2; NP; and membrane-anchored LASV GP1, GP2, and GPC polypeptides. The term polypeptide is used broadly herein to include peptide or protein or fragments thereof. By way of example and not limitation, a soluble LASV GP2 glycoprotein may comprise amino acid residues 260-427 of the LASV GPC protein (FIG. 2B), which residues represent the LASV GP2 protein lacking its transmembrane and intracellular domains. Also by way of example and not limitation, soluble LASV GP1 glycoprotein may comprise amino acid residues 59-259 of the LASV GPC protein. Also by way of example and not limitation, a membrane-anchored LASV GP2 glycoprotein may comprise amino acid residues 260-451 of the LASV GPC protein, which residues represent the LASV GP2 protein with its transmembrane domain.

Functional equivalents of these polypeptides are also intended to be encompassed by this invention. By way of example and not limitation, functionally equivalent polypeptides are those that possess one or more of the following characteristics: the ability to elicit antibodies (including, but not limited to, viral neutralizing antibodies) capable of recognizing native LASV polypeptides or the ability to interact with or bind antibodies found in serum of animals (including humans) that have been exposed to LASV (i.e., Lassa Fever convalescent patient sera).

Also intended to be encompassed are peptidomimetics, which include chemically modified peptides, peptide-like molecules containing non-naturally occurring amino acids, peptoids and the like, and retain the characteristics of the soluble or membrane-anchored LASV polypeptides provided herein. U.S. Pat. No. 7,144,856 (herein incorporated by reference in its entirety) describes compositions that can be employed to produce peptidomimetics of the present invention.

This invention further includes polypeptides or analogs thereof having substantially the same function as the polypeptides of this invention. Such polypeptides include, but are not limited to, a substitution, addition or deletion mutant of the inventive polypeptides. This invention also encompasses proteins or peptides that are substantially homologous to the polypeptides. A variety of sequence alignment software programs described herein above is available in the art to facilitate determination of homology or equivalence of any protein to a protein of the invention.

The term “analog” includes any polypeptide having an amino acid residue sequence substantially identical to a polypeptide of the invention in which one or more residues have been conservatively substituted with a functionally similar residue and which displays the functional aspects of the polypeptides as described herein. Examples of conservative substitutions include the substitution of one non-polar (hydrophobic) residue such as isoleucine, valine, leucine or methionine for another; the substitution of one polar (hydrophilic) residue for another such as beTWEEN arginine and lysine, beTWEEN glutamine and asparagine, beTWEEN glycine and serine; the substitution of one basic residue such as lysine, arginine or histidine for another; and the substitution of one acidic residue, such as aspartic acid or glutamic acid or another.

The phrase “conservative substitution” also includes the use of a chemically derivatized residue in place of a non-derivatized residue. “Chemical derivative” refers to a subject polypeptide having one or more amino acid residues chemically derivatized by reaction of a functional side group. Examples of such derivatized amino acids include for example, those amino acids in which free amino groups have been derivatized to form amine hydrochlorides, p-toluene sulfonyl groups, carbobenzoxy groups, t-butyloxycarbonyl groups, chloroacetyl groups or formyl groups. Also, the free carboxyl groups of amino acids may be derivatized to form salts, methyl and ethyl esters or other types of esters or hydrazides. Also, the free hydroxyl groups of certain amino acids may be derivatized to form O-acyl or O-alkyl derivatives. Also, the imidazole nitrogen of histidine may be derivatized to form N-imbenzylhistidine. Also included as chemical derivatives are those proteins or peptides which contain one or more naturally occurring amino acid derivatives of the twenty standard amino acids. For example, 4-hydroxyproline may be substituted for proline, 5-hydroxylysine may be substituted for lysine, 3-methylhistidine may be substituted for histidine, homoserine may be substituted for serine, and ornithine may be substituted for lysine. Polypeptides of the present invention also include any polypeptide having one or more additions and/or deletions of residues relative to the sequence of any one of the polypeptides whose sequence is described herein.

Two polynucleotide or polypeptide sequences are said to be “identical” if the sequence of nucleotides or amino acids in the two sequences is the same when aligned for maximum correspondence as described below. Comparisons beTWEEN two sequences are typically performed by comparing the sequences over a comparison window to identify and compare local regions of sequence similarity. A “comparison window” as used herein, refers to a segment of at least about 20 contiguous positions, usually 30 to about 75 contiguous positions, or 40 to about 50 contiguous positions, in which a sequence may be compared to a reference sequence of the same number of contiguous positions after the two sequences are optimally aligned.

Optimal alignment of sequences for comparison may be conducted using the Megalign program in the LASERGENE suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D. C. Vol. 5, Suppl. 3, pp. 345-358; Hem J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e. the window size) and multiplying the results by 100 to yield the percentage of sequence identity.

The analogs and homologs of all of the above-described inventive polypeptides and fragments thereof preferably have a sequence identity of about 95%, 96%, 97%, 98% or 99% with the polypeptides/fragments. However, analogs and homologs having a sequence identity of about 85%, 86%, 87%, 88%, 89%, 90%, 91%, 92%, 93% or 94% with the inventive polypeptides/fragments are also embodiments of the present invention. The present invention is also drawn to polynucleotides that encode analogs and homologs that have one of these levels of sequence identity with the inventive polypeptides.

A fragment of an inventive polypeptide preferably retains the same or similar function as the full-length version of the polypeptide. Preferred fragments of the above inventive peptides are about 50, 75, 100, 125, 150, 175, 200, 225, 250, 275, 300, 325, 350, 375, 400, 425, 450, 475, 500, 525 or 550 amino acid residues in length. As described above, analogs and homologs of such fragments are also embodiments of the present invention. Further, polynucleotides encoding these fragments and analogs/homologs thereof are invention embodiments.

As described above, the inventive polypeptides either comprise or consist of the soluble or membrane anchored forms of the LASV GP1, GP2, GPC, NP, fusion proteins thereof, homologs thereof and fragments thereof. The length of the proteins that comprise the inventive polypeptides are preferably about 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900 or 2000 amino acid residues. Embodiments of the invention are also drawn to polynucleotides encoding these polypeptides.

It should be understood that certain compositions of the present invention may comprise multiple components such as an appropriate pharmaceutical carrier. Various pharmaceutical carriers and other components for formulating the peptide for therapeutic use are described in U.S. Pat. Nos. 6,492,326 and 6,974,799, both of which are incorporated herein by reference in their entirety.

Although the inventive peptides may consist of a certain length of amino acids, such peptides may also incorporate non-amino acid entities such as functional groups. Functional groups can be complexed to the inventive peptides at the N-terminus via replacement of a hydrogen on the amine group, at the C-terminus via replacement of the hydroxyl on the carboxylic group, or at any reactive R group along the length of the peptide. Functional groups are well known in the art and are described, for example, in U.S. Patent Appl. Publ. No. 2006-0069027 A1, which is incorporated herein by reference in its entirety.

Finally, the fusion peptides of the present invention may comprise certain sequences, where one sequence consists of a particular LASV peptide, and the other sequences comprise non-LASV residues. In other words, where appropriate and stated as such, a fusion protein of the instant invention can be understood to comprise a non-LASV region fused to a particular LASV region to the exclusion of other LASV sequences. “Comprising” language used in this context thus would not read on, for example, full-length versions of an LASV protein for which a fragment thereof is in fusion.

### Expression Vectors

This invention also relates to expression vectors comprising at least one polynucleotide encoding a soluble or membrane-anchored protein of the invention. Expression vectors are well known in the art and include, but are not limited to viral vectors or plasmids. Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; all these applications and patents are herein incorporated by reference in their entirety), alphavirus-based vectors (e.g., Sindbis virus vectors, Semliki forest virus), Ross River virus, adeno-associated virus (AAV) vectors (see, e.g., PCT Publication Nos. WO 94/12649, WO 93/03769; WO 93/19191; WO 94/28938; WO 95/11984 and WO 95/00655; all these applications and patents are herein incorporated by reference in their entirety), vaccinia virus (e.g., Modified Vaccinia virus Ankara (MVA) or fowlpox), Baculovirus recombinant system and herpes virus.

Nonviral vectors, such as plasmids, are also well known in the art and include, but are not limited to, yeast- and bacteria-based plasmids.

Methods of introducing the vectors into a host cell and isolating and purifying the expressed protein are also well known in the art (e.g., Molecular Cloning: A Laboratory Manual, second edition, Sambrook, et al., 1989, Cold Spring Harbor Press). Examples of host cells include, but are not limited to, mammalian cells such as NS0 and CHO cells.

By way of example, vectors comprising the polynucleotide of the invention may further comprise a tag polynucleotide sequence to facilitate protein isolation and/or purification. Examples of tags include but are not limited to the myc-epitope, S-tag, his-tag, HSV epitope, V5-epitope, FLAG and CBP (calmodulin binding protein). Such tags are commercially available or readily made by methods known to the art.

The vector may further comprise a polynucleotide sequence encoding a linker sequence. Generally the linking sequence is positioned in the vector beTWEEN the soluble or membrane-anchored Lassa virus subunit protein polynucleotide sequence and the polynucleotide tag sequence. Linking sequences can encode random amino acids or can contain functional sites. Examples of linking sequences containing functional sites include but are not limited to, sequences containing the Factor Xa cleavage site, the thrombin cleavage site, or the enterokinase cleavage site.

By way of example, and not limitation, a soluble or membrane-anchored Lassa virus subunit protein may be generated as described herein using mammalian expression vectors in mammalian cell culture systems or bacterial expression vectors in bacterial culture systems. Examples of primers that may be used to amplify the desired ectodomain sequence from a Lassa virus cDNA template, include, but are not limited to, the primers in the Examples.

### Antibodies

Examples of antibodies encompassed by the present invention, include, but are not limited to, antibodies specific for soluble or membrane-anchored Lassa virus subunit proteins, antibodies that cross react with native Lassa virus antigens, and neutralizing antibodies. By way of example a characteristic of a neutralizing antibody includes the ability to block or prevent infection of a host cell. The antibodies of the invention may be characterized using methods well known in the art.

The antibodies useful in the present invention can encompass monoclonal antibodies, polyclonal antibodies, antibody fragments (e.g., Fab, Fab′, F(ab′)2, Fv, Fc, etc.), chimeric antibodies, bi-specific antibodies, heteroconjugate antibodies, single-chain fragments (e.g. ScFv), mutants thereof, fusion proteins comprising an antibody portion, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or of any other origin (including chimeric or humanized antibodies).

Methods of preparing monoclonal and polyclonal antibodies are well known in the art. Polyclonal antibodies can be raised in a mammal, for example, by one or more injections of an immunizing agent and, if desired an adjuvant. Examples of adjuvants include, but are not limited to, keyhole limpet hemocyanin (KLH), serum albumin, bovine thyroglobulin, soybean trypsin inhibitor, complete Freund adjuvant (CFA), and MPL-TDM adjuvant. The immunization protocol can be determined by one of skill in the art.

The antibodies may alternatively be monoclonal antibodies. Monoclonal antibodies may be produced using hybridoma methods (see, e.g., Kohler, B. and Milstein, C. (1975) Nature 256:495-497 or as modified by Buck, D. W., et al., In Vitro, 18:377-381 (1982).

If desired, the antibody of interest may be sequenced and the polynucleotide sequence may then be cloned into a vector for expression or propagation. The sequence encoding the antibody of interest may be maintained in the vector in a host cell, and the host cell can then be expanded and frozen for future use. In an alternative embodiment of the invention, the polynucleotide sequence may be used for genetic manipulation to “humanize” the antibody or to improve the affinity, or other characteristics of the antibody (e.g., genetically manipulate the antibody sequence to obtain greater affinity to the soluble or membrane-anchored Lassa virus subunit protein and/or greater efficacy in inhibiting the fusion of Lassa virus to the host cell).

The antibodies may also be humanized by methods known in the art (See, for example, U.S. Pat. Nos. 4,816,567; 5,807,715; 5,866,692; 6,331,415; 5,530,101; 5,693,761; 5,693,762; 5,585,089; and 6,180,370; all these patents are herein incorporated by reference in their entirety). In yet another alternative, fully human antibodies may be obtained by using commercially available mice that have been engineered to express specific human immunoglobulin proteins.

In another alternative embodiment of the invention, antibodies may be made recombinantly and expressed using any method known in the art. By way of example, antibodies may be made recombinantly by phage display technology. See, for example, U.S. Pat. Nos. 5,565,332; 5,580,717; 5,733,743; and 6,265,150 (all these patents are herein incorporated by reference in their entirety); and Winter et at., Annu. Rev. Immunol. 12:433-455 (1994). Alternatively, phage display technology (McCafferty et al., Nature 348:552-553 (1990)) can be used to produce human antibodies and antibody fragments in vitro. Phage display can be performed in a variety of formats; for review, see Johnson, Kevin S. and Chiswell, David J., Current Opinion in Structural Biology 3:564-57 1 (1993). By way of example, a soluble or membrane-anchored Lassa virus subunit protein as described herein may be used as an antigen for the purposes of isolating recombinant antibodies by these techniques.

Antibodies may be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or transgenic milk. Methods for expressing antibodies recombinantly in plants or milk have been disclosed. See, for example, Peeters, et al. Vaccine 19:2756 (2001); Lonberg, N. and D. Huszar Int. Rev. Immunol 13:65 (1995); and Pollock, et al., J. Immunol. Methods 231:147 (1999). Methods for making derivatives of antibodies (e.g. humanized and single-chain antibodies, etc.) are known in the art.

The antibodies of the invention can be bound to a carrier by conventional methods for use in, for example, isolating or purifying a soluble or membrane-anchored Lassa virus subunit protein or detecting Lassa virus subunit proteins, antigens, or particles in a biological sample or specimen. Alternatively, by way of example, the neutralizing antibodies of the invention may be administered as passive immunotherapy to a subject infected with or suspected of being infected with Lassa virus. A “subject,” includes but is not limited to humans, simians, farm animals, sport animals, and pets. Veterinary uses are also encompassed by the invention.

### Diagnostics

The soluble or membrane-anchored Lassa virus subunit proteins and/or antibodies of the invention may be used in a variety of immunoassays for Lassa virus and other arenaviruses. The recombinantly expressed soluble or membrane-anchored Lassa virus subunit proteins of the invention can be produced with high quality control and are suitable as antigens for the purposes of detecting antibody in biological samples. The antibodies of the invention, e.g., those raised against or panned by the soluble or membrane-anchored Lassa virus subunit proteins of the invention, can also be produced with high quality control and are suitable as reagents for the purposes of detecting antigen in biological samples. By way of example and not limitation, a soluble or membrane-anchored Lassa virus subunit protein or combinations thereof could be used as antigens in an enzyme-linked immunosorbent assay (ELISA) assay to detect antibody in a biological sample from a subject. Also by way of example, and not limitation, antibodies of the invention could be used as reagents in an ELISA assay to detect Lassa antigen in a biological sample from a subject.

### Vaccines

This invention also relates to vaccines for Lassa virus and other arenaviruses. In one aspect the vaccines are DNA-based vaccines. One skilled in the art is familiar with administration of expression vectors to obtain expression of an exogenous protein in vivo. See, e.g., U.S. Pat. Nos. 6,436,908; 6,413,942; and 6,376,471 (all these patents are herein incorporated by reference in their entirety). Viral-based vectors for delivery of a desired polynucleotide and expression in a desired cell are well known in the art and non-limiting examples are described herein.

Administration of expression vectors includes local or systemic administration, including injection, oral administration, particle gun or catheterized administration, and topical administration. Targeted delivery of therapeutic compositions containing an expression vector or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Non-viral delivery vehicles and methods can also be employed, including but not limited to, polycationic condensed DNA linked or unlinked to killed adenovirus alone (see, e.g., Cunel, Hum. Gene Ther. (1992) 3:147); ligand-linked DNA (see, e.g., Wu, J. Biol. Chem. (1989) 264:16985); eukaryotic cell delivery vehicles (see, e.g., U.S. Pat. No. 5,814,482; PCT Publication Nos. WO 95/07994; WO 96/17072; WO 95/30763; and WO 97/42338); and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796, WO 94/23697, WO 9 1/14445; and EP 0524968. Additional approaches are described in Philip, Mol. Cell Biol. (1994) 14:2411, and in Woffendin, Proc. Natl. Acad. Sci. (1994) 91:1581.

For human administration, the codons comprising the polynucleotide encoding a soluble LASV glycoprotein may be optimized for human use, a process which is standard in the art.

In another aspect of the invention, a soluble Lassa subunit protein or combination thereof is used as a subunit vaccine. The soluble Lassa subunit protein or combination thereof may be administered by itself or in combination with an adjuvant. Examples of adjuvants include, but are not limited, aluminum salts, water-in-soil emulsions, oil-in-water emulsions, saponin, QuilA and derivatives, iscoms, liposomes, cytokines including gamma-interferon or interleukin 12, DNA (e.g. unmethylated poly-CpG), microencapsulation in a solid or semi-solid particle, Freunds complete and incomplete adjuvant or active ingredients thereof including muramyl dipeptide and analogues, DEAE dextrarilmineral oil, ALHYDROGEL, AUSPHARM adjuvant, and ALGAMMULIN.

The subunit vaccine comprising a Lassa subunit protein or combinations thereof can be administered orally or by any parenteral route such as intravenously, subcutaneously, intraarterially, intramuscularly, intracardially, intraspinally, intrathoracically, intraperitoneally, intraventricularly, sublingually, and/or transdermally.

Dosage and schedule of administration can be determined by methods known in the art. Efficacy of the soluble Lassa subunit protein or combinations thereof as a vaccine for Lassa virus or related arenaviruses may also be evaluated by methods known in the art.

### Pharmaceutical Compositions

The polynucleotides, polypeptides, and antibodies of the invention can further comprise pharmaceutically acceptable carriers, excipients, or stabilizers known in the art (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are non-toxic to recipients at the employed dosages and concentrations, and may comprise buffers such as phosphate, citrate, and other organic acids; antioxidants including ascorbic acid and methionine; preservatives (e.g. octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, marmose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The compositions used in the methods of the invention generally comprise, by way of example and not limitation, an effective amount of a polynucleotide or polypeptide (e.g., an amount sufficient to induce an immune response) of the invention or antibody of the invention (e.g., an amount of a neutralizing antibody sufficient to mitigate infection, alleviate a symptom of infection and/or prevent infection).

The pharmaceutical composition of the present invention can further comprise additional agents that serve to enhance and/or complement the desired effect. By way of example, to enhance the immunogenicity of a soluble Lassa subunit protein of the invention being administered as a subunit vaccine, the pharmaceutical composition may further comprise an adjuvant. Examples of adjuvants are provided herein.

Also by way of example and not limitation, if a soluble Lassa subunit polypeptide of the invention is being administered to augment the immune response in a subject infected with or suspected of being infected with Lassa virus and/or if antibodies of the present invention are being administered as a form of passive immunotherapy, the composition can further comprise other therapeutic agents (e.g., anti-viral agents).

### Kits

The invention also provides kits for use in the instant methods. Kits of the invention include one or more containers comprising by way of example, and not limitation, polynucleotides encoding a soluble or membrane-anchored Lassa virus subunit protein or combinations thereof and/or antibodies of the invention and instructions for use in accordance with any of the methods of the invention described herein.

Generally, these instructions comprise a description of administration or instructions for performance of an assay. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (e.g. the container may be an intravenous solution bag or a vial having a stopper pierceable by a hypodermic injection needle). The container may also have a sterile access port (e.g. the container may be an intravenous solution bag or a vial having a stopper pierceable by a hypodermic injection needle). Kits may optionally provide additional components such as buffers and interpretive information. Normally, the kit comprises a container and a label or package insert(s) on or associated with the container.

### EXAMPLES

### Example 1

### LASV Infection, cDNA Synthesis, and PCR Amplification of LASV Genes

Vero cells were infected with LASV strain Josiah at a multiplicity of infection (MOI) of 0.1. Briefly, virus was diluted in complete Eagle's modified essential media (cEMEM) to a final volume of 2.0 mL, then added to confluent cells in a T-75 flask and incubated for 1 hour (h) at 37° C., with 5% CO₂ and periodic rocking (complete media refers to media containing animal serum). Subsequently, 13 mL of cEMEM was added, and the culture was incubated in a similar manner for 96 h. To prepare total cellular RNA, the cell culture medium was replaced with 2 mL of TRIZOL LS reagent (Invitrogen), and total RNA was purified according to the manufacturer's specifications.

Using the PROTOSCRIPT FIRST STRAND cDNA Synthesis Kit (New England BioLabs), 100 ng of total cellular RNA per reaction was transcribed into cDNA, as outlined in the manufacturer's protocol. The PHUSION HIGH-FIDELITY Polymerase Chain Reaction (PCR) MASTERMIX (New England Biolabs) was used in all amplifications of LASV gene sequences. PCR parameters were determined based on the melting temperature (Tm) for each oligonucleotide set. LASV GP1 and GP2 genes were amplified using the following cycling conditions: 98° C. for one 15 second (sec) cycle and then 35 repeated cycles of 98° C. for 5 sec, 59° C. for 10 sec, and 72° C. for 15 sec, followed by a final extension at 72° C. for 5 minutes (min). LASV NP was amplified using the following cycling conditions: 98° C. for one 30 sec cycle and then 35 repeated cycles of 98° C. for 10 sec, 59° C. for 15 sec, and 72° C. for 30 sec, followed by a final extension at 72° C. for 5 min.

Table 1 outlines each of the nucleotide sequences of the oligonucleotide primers used in the amplification of LASV genes for expression in bacterial cell systems. The ectodomain of the LASV GP1 gene, lacking a signal sequence and the N-terminal methionine (N-Met), was amplified using a 41-mer forward oligonucleotide primer (5′ GP1 bac), which contained a Bam HI Restriction Endonuclease (REN) site and comprised a sequence encoding the N-terminal 8 amino acids (a.a.) of the mature GP1 protein beyond the known SPase cleavage site, and a 49-mer reverse oligonucleotide primer (3′ GP1 bac), which contained a Hind III REN site, as well as two termination codons, and comprised a sequence encoding the C-terminal 10 a.a. of the mature GP1 protein. The 5′ and 3′ GP1 bac primers amplify the nucleotides encoding a.a. residues 59-259 of the LASV GPC, which residues represent all a.a. of LASV GP1. The ectodomain of the LASV GP2 gene was amplified using a 38-mer forward oligonucleotide primer (5′ GP2 bac), which contained a Bam HI REN site and comprised a sequence encoding the N-terminal 7 a.a. of the mature GP2 protein beyond the known SKI1/S1P protease cleavage site, and a 40-mer reverse oligonucleotide primer (3′ GP2 bac), which contained a Hind III REN site, as well as two termination codons, and comprised a sequence encoding the C-terminal 7 a.a. of the GP2 protein preceding the start of the native transmembrane (TM) anchor domain. The 5′ and 3′ GP2 bac primers amplify the nucleotides encoding a.a. residues 260-426 of the LASV GPC, which residues represent LASV GP2 lacking its TM and IC domains. The LASV NP gene sequence was amplified using a 77-mer forward oligonucleotide primer (5′ NP bac), which contained an Eco RI REN site and comprised a sequence encoding the N-terminal 22 a.a. of the polypeptide without the N-Met, and a 43-mer reverse oligonucleotide primer (3′ NP bac), which contained a Hind III REN site, as well as two termination codons, and comprised a sequence encoding the C-terminal 8 a.a. of the NP protein.

TABLE 1

| LASV Gene | | |
|---|---|---|
| Amplified | LASV Primer | Oligonucleotide Primer Sequence |
| GP1 | 5′ GP1 bac | TTTCAGAATTCGGATCCACCAGTCTTTATAA |
| | | AGGGGTTTAT (SEQ ID NO: 12) |
| | 3′ GP1 bac | GGTACCAAGCTTTCAGTCATAGCAATCTTCT |
| | | ACTAATATAAATATCTCT (SEQ ID NO: 13) |
| GP2 | 5′ GP2 bac | TTTCAGAATTCGGATCCGGCACATTCACATG |
| | | GACACTG (SEQ ID NO: 14) |
| | 3′ GP2 bac | GGTACCAAGCTTTCAGCTATGTCTTCCCCTG |
| | | CCTCTCCAT (SEQ ID NO: 15) |
| NP | 5′ NP bac | TTTCAGAATTCAGTGCCTCAAAGGAAATAA |
| | | AATCCTTTTTGTGGACACAATCTTTGAGGAG |
| | | GGAATTATCTGGTTAC (SEQ ID NO: 16) |
| | 3′ NP bac | GGTACCAAGCTTTCAGTTACAGAACGACTC |
| | | TAGGTGTCGATGT (SEQ ID NO: 17) |

Note.
REN sites are underlined, and stop codons (TCA, CTA, TTA; in complementary orientation) are in bold print.

Table 2 outlines each of the nucleotide sequences of the oligonucleotide primers used in the amplification of LASV genes for expression in mammalian cell systems. The LASV GPC open reading frame (ORF) was amplified using a 36-mer forward oligonucleotide primer (5′ GPC), which contained an Nhe I REN site and comprised a sequence encoding the N-terminal 9 a.a. of the GPC signal peptide (SP), and a 40-mer reverse oligonucleotide primer (3′ GPC), which contained a Hind III REN site, as well as two termination codons and comprised a sequence encoding the C-terminal 7 a.a. of the intracellular domain (IC) of GP2 (GP2-IC).

TABLE 2

| LASV Gene | | |
|---|---|---|
| Amplifed | LASV Primer | Oligonucleotide Primer Sequence |
| GPC | 5′ GPC | GTAGCTAGCATGGGACAAATAGTGACATTCTTCCAG |
| | | (SEQ ID NO: 18) |
| | 3′ GPC | GGTACCAAGCTTTCAGTCATCTCTTCCATTTCACAGG |
| | | CAC (SEQ ID NO: 19) |
| GP1-TM | 5′ GPC | GTAGCTAGCATGGGACAAATAGTGACATTCTTCCAG |
| | | (SEQ ID NO: 18 |
| | 3′ GP1-TM | GGTACCAAGCTTTCAGTCATGGTATTTTGACTAGGT |
| | | GAAGGAAGATGCTAATAAGATAGAAACTTGTGCTGA |
| | | ACACAAAGAGGTCAACTAGACCCAATGGTAGCAATC |
| | | TTCTACTAATATAAATATCTCT (SEQ ID NO: 20) |
| sGP1 | 5′ GPC | GTAGCTAGCATGGGACAAATAGTGACATTCTTCCAG |
| | | (SEQ ID NO: 18) |
| | 3′ GP1 bac | GGTACCAAGCTTTCAGTCATAGCAATCTTCTACTAA |
| | | TATAAATATCTCT (SEQ ID NO: 13) |
| sGP1-FLAG | 5′ GPC | GTAGCTAGCATGGGACAAATAGTGACATTCTTCCAG |
| | | (SEQ ID NO: 18) |
| | 3′ sGP1-FLAG | CGATAAGCTTTCAGTCAGCCCTTGTCGTCGTCGTCCT |
| | | TGTAGTCTAGCAATCTTCTACTAATATA |
| | | (SEQ ID NO: 21) |
| sGP1-h λ | 5′ sGP1-h λ LC | GATCGCTAGCGCCGCCACCATGGGCTGGAGCTGCAT |
| LC | | CATCCTGTTCCTGGTGGCCACCGCCACCGGCGTGCAC |
| | | AGCACCAGTCTTTATAAAGGGGTT (SEQ ID NO: 22) |
| | 3′ GP1 bac | GGTACCAAGCTTTCAGTCATAGCAATCTTCTACTAA |
| | | TATAAATATCTCT (SEQ ID NO: 13) |
| sGP1-h HC | 5′ sGP1-h HC | GATCGCTAGCGCCGCCACCATGGGCTGGAGCTGCAT |
| | | HCCATCCTGTTCCTGGTGGCCACCGCCACCGGCGTGCAC |
| | | AGCACCAGTCTTTATAAAGGGGTT (SEQ ID NO: 23) |
| | 3′ GP1 bac | GGTACCAAGCTTTCAGTCATAGCAATCTTCTACTAA |
| | | TATAAATATCTCT (SEQ ID NO: 13) |
| sGP2-h λ | 5′ sGP2-h λ LC | AAGCTGGCTAGCCACCATGGCCTGGTCTCCTCTCCTC |
| LC | | CTCACTCTCCTCGCTCACTGCACAGGGTCCTGGGCCC |
| | | AGGGCACATTCACATGGACACTG (SEQ ID NO: 24) |
| | 3′ GP2 bac | GGTACCAAGCTTTCAGCTATGTCTTCCCCTGCCTCTC |
| | | CAT (SEQ ID NO: 15) |
| sGP2-h HC | 5′ sGP2-h HC | GATCGCTAGCGCCGCCACCATGGGCTGGAGCTGCAT |
| | | CATCCTGTTCCTGGTGGCCACCGCCACCGGCGTGCAC |
| | | AGCGGCACATTCACATGGACACTG (SEQ ID NO: 25) |
| | 3′ GP2 bac | GGTACCAAGCTTTCAGCTATGTCTTCCCCTGCCTCTC |
| | | CAT (SEQ ID NO: 15) |
| GP2-TM-h λ | 5′ GP2-h λ LC | AAGCTGGCTAGCCACCATGGCCTGGTCTCCTCTCCTC |
| LC | | CTCACTCTCCTCGCTCACTGCACAGGGTCCTGGGCCC |
| | | AGGGCACATTCACATGGACACTG (SEQ ID NO: 26) |
| | 3′ GP2-TM | GGTACCAAGCTTTCAGTCATGGTATTTTGACTAGGT |
| | | G AAGGAA (SEQ ID NO: 27) |
| GP2-TM-h | 5′ GP2-h HC | GATCGCTAGCGCCGCCACCATGGGCTGGAGCTGCAT |
| HC | | CATCCTGTTCCTGGTGGCCACCGCCACCGGCGTGCAC |
| | | AGCGGCACATTCACATGGACACTG (SEQ ID NO: 28) |
| | 3′ GP2-TM | GGTACCAAGCTTTCAGTCATGGTATTTTGACTAGGT |
| | | G AAGGAA (SEQ ID NO: 27) |
| sGP2-FLAG | 5′ sGP2-h HC | GATCGCTAGCGCCGCCACCATGGGCTGGAGCTGCAT |
| | | CATCCTGTTCCTGGTGGCCACCGCCACCGGCGTGCAC |
| | | AGCGGCACATTCACATGGACACTG (SEQ ID NO: 25) |
| | 3′ sGP2-FLAG | CGATAAGCTTTCAGTCAGCCCTTGTCGTCGTCGTCCT |
| | | TGTAGTCTGTCTTCCCCTGCCTCTCCAT |
| | | (SEQ ID NO: 29) |

Note.
REN sites are underlined, start codons (ATG) are double-underlined, and stop codons (TCA, CTA; in complementary orientation) are in bold print. The sequence encoding the FLAG-tag domain is italicized.

Five different versions of LASV GP1 were generated for these studies, including one TM-anchored protein, GP1-TM, and four soluble (s) proteins, sGP1, sGP1-FLAG, sGP1-h λ LC, and sGP1-h HC. GP1-TM is comprised of the native GPC SP through the last a.a. of the mature GP1 protein and is fused to a sequence identical to the LASV GP2 TM domain (GP2-TM), including an additional 3 a.a. from the predicted GP2-IC. The sequence encoding GP1-TM was amplified using the same forward oligonucleotide primer (5′ GPC) used for amplification of the GPC gene, as outlined above, and a 130-mer reverse oligonucleotide primer (3′ GP1-TM), which contained a Hind III REN site, as well as two termination codons, and comprised a sequence encoding the C-terminal 10 a.a. of the mature GP1 protein fused to the 24 a.a. sequence of GP2-TM plus an additional 3 a.a. of the GP2-IC domain. These versions of GP1 were designed to contain either the native GPC SP or the SP of a human IgG λ light chain (h λ LC) or human IgG heavy chain (h HC). The two sGP1 proteins, sGP1 and sGP1-FLAG, each contained the native GPC SP through the last a.a. of the mature GP1 protein and differed only in the presence of a FLAG-tag sequence (DYKDDDDKG, SEQ ID NO:30) on the C-terminus of the latter protein, which facilitated purification through a FLAG affinity resin. The sequence encoding sGP1 was amplified using the same forward oligonucleotide primer (5′ GPC) used for amplification of GPC, as outlined above, and the same reverse oligonucleotide primer (3′ GP1 bac) used for the aforementioned amplification of the GP1 gene for bacterial expression. Amplification of the sGP1-FLAG gene was performed using the forward primer 5′ GPC and a 65-mer reverse oligonucleotide primer (3′ sGP1-FLAG), which contained a Hind III REN site, as well as two termination codons, and comprised a sequence encoding the FLAG-tag domain fused to the C-terminal 7 a.a. of mature GP1. The sequence encoding sGP1-h λ LC was amplified using a 97-mer forward oligonucleotide primer (5′ sGP1-h λ LC), which contained an Nhe I REN site and comprised a sequence encoding an optimized Kozak translation initiation site and a h λ LC sequence fused to the N-terminal 7 a.a. of mature GP1. The reverse oligonucleotide primer (3′ GP1 bac) used for amplification of sGP1-h λ LC was the same as that used in the amplification of the GP1 gene for bacterial expression. The sequence coding for sGP1-h HC was amplified using a 97-mer forward oligonucleotide primer (5′ sGP1-h HC), which contained an Nhe I REN site and comprised a sequence encoding an optimized Kozak translation initiation site and a h λ LC fused to the N-terminal 7 a.a. of mature GP1. The reverse oligonucleotide primer (3′ GP1 bac) used for amplification of this gene was the same as that used in the amplification of the GP1 gene for bacterial expression.

Five different versions of LASV GP2 were generated for these studies, including two TM-anchored proteins, GP2-TM-h HC and GP2-TM-h λ LC, and three soluble (s) proteins, sGP2-h HC, sGP2-h λ LC, and sGP2-FLAG. GP2-TM-h HC comprised the h HC fused to the N-terminus of the GP2 ORF, starting at a.a. 260, and included the GP2-TM and an additional 3 a.a. from the predicted GP2-IC. The sequence encoding GP2-TM-h HC was amplified using a 97-mer forward oligonucleotide primer (5′ sGP2-h HC), which contained an Nhe I REN site and comprised a sequence encoding an optimized Kozak translation initiation site and h HC sequence fused to the N-terminal 7 a.a. of mature GP2, and a 43-mer reverse oligonucleotide primer (3′ GP2-TM), which contained a Hind III REN site, as well as two termination codons, and comprised a sequence encoding the C-terminal 8 a.a. of GP2-TM, including an additional 3 a.a. from the predicted GP2-IC. GP2-TM-h λ LC comprised the h λ LC fused to the N-terminus of the GP2 ORF, starting at a.a. 260, and included GP2-TM and an additional 3 a.a. from the predicted GP2-IC. The sequence encoding GP2-TM-h λ LC was amplified using a 97-mer forward oligonucleotide primer (5′ sGP2-h λ LC), which contained an Nhe I REN site and comprised a sequence encoding an optimized Kozak translation initiation site and a h λ LC sequence fused to the N-terminal 7 a.a. of mature GP2. The reverse oligonucleotide primer (3′ GP2-TM) used for amplification of this gene was the same as that used in the amplification of the GP2-TM-h HC gene. The same forward oligonucleotide primer (5′ sGP2-h HC) used in the amplification of GP2-TM-h HC was also used to amplify sGP2-h HC. The reverse oligonucleotide primer (3′ GP2 bac) used for amplification of this gene was the same as that used in the amplification of the GP2 gene for bacterial expression. The same forward oligonucleotide primer (5′ sGP2-h λ LC) that was used in the amplification of GP2-TM-h λ LC was also used to amplify sGP2-h λ LC. The reverse oligonucleotide primer (3′ GP2 bac) used for amplification of this gene was the same as that used in the amplification of the GP2 gene for bacterial expression. Amplification of sGP2-FLAG was performed with the forward oligonucleotide primer 5′ sGP2-h HC, which was the same as that used to amplify GP-TM-h HC, and a 65-mer reverse oligonucleotide primer (3′ sGP2-FLAG), which contained a Hind III REN site, as well as two termination codons, and comprised a sequence encoding the FLAG-tag domain fused to the C-terminal 7 a.a. of GP2 preceding the TM domain.

### Example 2

### Cloning LASV Genes for Expression in Bacterial and Mammalian Cell Systems

FIG. 2A summarizes the strategy used to clone LASV GP1, GP2, and NP gene sequences into vectors pMAL-p2x and -c2x for expression in bacteria. As outlined in Table 3, initial pilot expression studies were performed with vectors pMAL-p2x:GP1, pMAL-p2x:GP2, and pMAL-p2x:NP in the Rosetta 2(DE3) E. coli strain.

TABLE 3

| Recombinant Plasmid | LASV Gene | Expression System |
|---|---|---|
| pMAL-p2X: GP1 | GP1 | Rosetta 2(DE3) |
| pMAL-p2X: GP2 | GP2 | Rosetta 2(DE3) |
| pMAL-p2X: NP | NP | Rosetta 2(DE3) |
| pMAL-c2X: GP1 | GP1 | Rosetta Gami 2 |
| pMAL-c2X: GP2 | GP2 | Rosetta Gami 2 |
| pMAL-c2X: NP | NP | Rosetta 2(DE3) |

Subsequent experiments used vectors pMAL-c2x:GP1, pMAL-c2x:GP2, and pMAL-c2x:NP, with the former two constructs expressed in E. coli Rosetta Gami 2 cells and the latter in E. coli Rosetta 2(DE3) cells. The strategy for cloning LASV GPC, all versions of GP1, and all GP2 gene sequences into mammalian expression vectors is outlined in FIG. 2B. Table 4 summarizes the recombinant plasmids and applicable cell lines for mammalian expression. DNA was manipulated by standard techniques (Sambrook et al.), and all recombinant plasmids outlined in Tables 3 and 4 were initially engineered and propagated in E. coli DH5α.

TABLE 4

| | | Expression |
|---|---|---|
| Recombinant Plasmid | LASV Gene | System |
| pcDNA3.1/Zeo(+) - GPC | GPC | 293T/17, |
| pcDNA3.1/Zeo(+): intA - GPC | GPC | NS0 |
| pcDNA3.1/Zeo(+) - GP1-TM | GP1-TM | |
| pcDNA3.1/Zeo(+): intA - GP1-TM | GP1-TM | |
| pcDNA3.1/Zeo(+) - GP2-TM-h HC | GP2-TM | |
| pcDNA3.1/Zeo(+): intA - GP2-TM-h HC | GP2-TM | |
| pcDNA3.1/Zeo(+) - GP2-TM-h λ LC | GP2-TM | |
| pcDNA3.1/Zeo(+): intA - GP2-TM-h λ LC | GP2-TM | |
| pBFdhfr.1 - sGP1 | sGP1 | 293T/17, |
| pBFdhfr.2: intA - sGP1 | sGP1 | CHO DG44 |
| pBFdhfr.1 - sGP1- h HC | sGP1 | |
| pBFdhfr.2: intA - sGP1- h HC | sGP1 | |
| pBFdhfr.1 - sGP1- h λ LC | sGP1 | |
| pBFdhfr.2: intA - sGP1- h λ LC | sGP1 | |
| pBFdhfr.1 - sGP1-FLAG | sGP1-FLAG | |
| pBFdhfr.2: intA - sGP1-FLAG | sGP1-FLAG | |
| pBFdhfr.1 - sGP2- h HC | sGP2 | |
| pBFdhfr.2: intA - sGP2- h HC | sGP2 | |
| pBFdhfr.1 - sGP2- h λ LC | sGP2 | |
| pBFdhfr.2: intA - sGP2- h λ LC | sGP2 | |
| pBFdhfr.1 - sGP2-FLAG | SGP2-FLAG | |
| pBFdhfr.2: intA - sGP2-FLAG | SGP2-FLAG | |

### Example 3

### Optimization of Recombinant LASV Protein Expression in Bacteria

Small scale pilot experiments were performed with each construct to determine optimal expression conditions for each maltose binding protein (MBP)-LASV fusion protein. Briefly, 50 mL shaker flask cultures of transformed E. coli were grown in cLB at 22° C., 30° C., and 37° C. to an A₆₀₀=0.5-0.6. Each culture was next split into three flasks and induced with isopropyl 13-D-1-thiogalactopyranoside (IPTG) to final concentrations of 0.03, 0.15 and 0.3 mM. Cultures were then grown under induction conditions for 2 h. Subsequently, periplasmic and cytoplasmic fractions were prepared by osmotic shock of E. coli transformed with pMAL-p2x-based vectors and by generation of whole cell lysates of E. coli transformed with pMAL-c2x-based vectors, respectively. MBP-LASV fusion proteins were captured from each fraction on amylose resin (New England BioLabs) and then analyzed by reducing Sodium Dodecyl Sulfate-Polyacrylamide Gel Electrophoresis (SDS-PAGE). Using optimal temperature and IPTG parameters established in the studies above, a time course study was carried out to further maximize total fusion protein yields. SDS-PAGE analysis was performed on LASV-MBP fusion proteins captured on amylose resin from samples harvested at 2, 3, and 4 h after induction.

### Example 4

### Scheme for Small-Scale Purification of Recombinant LASV Proteins Expressed in Bacteria

LASV-MBP fusion proteins were purified from whole cell lysates of E. coli transformed with pMAL-c2X-based vectors by capture on amylose resin followed by Factor Xa cleavage, according to the manufacturer's instructions (New England BioLabs). The addition of 1 mM dithiothreitol (DTT) was necessary to prevent aggregation and precipitation of protein before and during Factor Xa cleavage of LASV GP1-MBP and GP2-MBP fusion proteins. Moreover, addition of 0.03 to 0.05% SDS was required for efficient Factor Xa cleavage of both LASV GP1-MBP and LASV GP2-MBP fusion proteins. Briefly, cleaved LASV proteins were separated from MBP and other contaminants using a SUPERDEX 200 Prep Grade size exclusion column (Amersham Biosciences, Pittsburgh, Pa.). To prevent aggregation, 30 mM 2-(N-morpholino)ethanesulphonic acid (MES) buffer containing 0.1% (w/v) SDS was required for size-exclusion chromatography (SEC) purification of Factor Xa-treated GP2-MBP fusion protein; whereas, SEC purification of Factor Xa-treated GP1-MBP fusion protein required 30 mM MES buffer containing 5 mM DTT and 0.1% (w/v) SDS. LASV NP-MBP was cleaved with Factor Xa alone and was purified by SEC using 1×PBS, pH 7.4. These conditions were subsequently applied to the large-scale purification schemes of the respective LASV proteins.

### Example 5

### Large-Scale Culture and Purification of Recombinant LASV Proteins Expressed in Bacteria

To purify LASV NP, a 3-L shaker flask culture of pMAL-c2x:NP-transformed Rosetta 2(DE3) cells was grown in cLB to an A₆₀₀=0.5-0.6 at 30° C. and then induced with a final IPTG concentration of 0.03 mM. After incubation at 30° C. for 4 h, cells were harvested by centrifugation for 10 min at ˜13,000 g. The cell paste was frozen at −20° C. and subsequently thawed and resuspended in nine volumes of lysis buffer (20 mM TrisHCl, 200 mM NaCl, 10 mM EDTA, pH 8.0). Next, a bacterial protease inhibitor cocktail (Sigma) and lysozyme (Pierce Biotechnology, Rockford, Ill.) were added at concentrations of 4 mL per gram and 40 mg per gram of wet cell paste, respectively, after which the suspension was incubated at 37° C. with agitation. After 30 min, 1/10 volume of 1 M MgSO₄ and 50 μL of 2000 U/mL DNase I (Roche, Nutley, N.J.) per gram wet cell paste were added. The solution was incubated for an additional 30 min at 37° C. and then centrifuged at 13,000 g for 60 min at 4° C. The resulting supernatant was further clarified by 0.2-μm filtration, then diluted two-fold with lysis buffer and applied to a 1.6×10 cm amylose column at 75 cm/h. The column was washed with five column volumes of equilibration buffer (20 mM TrisHCl, 200 mM NaCl, pH 7.4), and the fusion protein eluted with equilibration buffer containing 10 mM maltose. For every A₂₈₀=1 of fusion protein, 20 μL of 1 mg/mL Factor Xa (Novagen) was added. The reaction mixture was then incubated overnight at 4° C. and subsequently clarified by low speed centrifugation followed by 0.2-μm filtration. The solution was loaded onto a 2.6×70 cm SUPERDEX 200 Prep Grade size exclusion column (Amersham Biosciences) in 6 mL aliquots and eluted at 60 cm/h with 1×PBS, pH 7.4. LASV NP-containing fractions were pooled and concentrated using an AMICON stirred cell unit fitted with a 10,000 NMWL (nominal molecular weight limit) ultrafiltration membrane (Millipore, Billerica, Mass.) at 20 psig nitrogen. Purified LASV NP was sterile-filtered using a 0.2-μm MILLEX GV syringe filter (Millipore), aliquoted and stored at −20° C.

For purification of LASV GP1, a 10-L culture of pMAL-c2x:GP1-transformed Rosetta-gami 2 cells was grown in semi-defined batch medium at 37° C. using a New Brunswick Scientific Fermentor (Edison, N.J.). When the density of the culture reached A₆₀₀=5.3, the temperature was reduced to 22° C., and IPTG was added to a final concentration of 1.58 mM. During the bacterial culture incubation, dissolved oxygen was set at 70%, and the culture was supplemented with 50% glucose to maintain glucose levels beTWEEN 0.2-2 g/L. At 4 h post-induction (A₆₀₀=8.9), cells were harvested by centrifugation for 10 min at ˜13,000 g. The resulting cell paste was frozen at −80° C. and subsequently thawed and resuspended in nine volumes of lysis buffer (20 mM TrisHCl, 200 mM NaCl, 10 mM EDTA, 1 mM DTT, pH 8.0). As described above for NP purification, bacterial protease inhibitor cocktail and lysozyme were added to the suspension, and the reaction was incubated at 37° C. with agitation. After 45 min, 1/10 volume of 1 M MgSO₄ and 50 μL 2000 U/mL DNase I (Roche) per gram wet cell paste were added. The solution was incubated for an additional 30 min at 37° C. and then centrifuged at 15,000 g for 60 min at 4° C. The supernatant was further clarified by 0.2-μm filtration and applied to a 2.6×12 cm amylose column at 75 cm/hr. The column was washed with five column volumes of equilibration buffer (20 mM TrisHCl, 200 mM NaCl, 1 mM EDTA, 1 mM DTT, pH 7.4), and the fusion protein eluted with equilibration buffer containing 10 mM maltose. SDS was added to a final concentration of 0.05% (w/v), followed by 20 μL of 1 mg/mL Factor Xa per A₂₈₀=1 of amylose column eluate. The reaction mixture was then incubated overnight at 4° C. The solution was then concentrated two-fold with a CENTRIPLUS YM-3 unit (Millipore) at 2,000 g at room temperature (RT), followed by the addition of DTT to a final concentration of 5 mM. Subsequently, the solution was loaded onto a 2.6×70 cm SUPERDEX 200 Prep Grade size exclusion column (Amersham Biosciences) in ˜3 mL aliquots and eluted with 30 mM MES, 154 mM NaCl, 0.1% SDS, 5 mM DTT, pH 6.7, at 30 cm/h. The fractions containing full-length GP1 were pooled separately from fractions containing GP1 fragments. Both GP1 pools were concentrated using an AMICON stirred cell unit fitted with a 3,000 NMWL ultrafiltration membrane (Millipore) at 55 psig nitrogen. To remove high molecular weight contaminants and DTT, full-length LASV GP1 was re-run on the SUPERDEX 200 column with 30 mM MES, 154 mM NaCl, 0.1% SDS, pH 6.7. The GP1 fragment pool was dialyzed in SEC buffer using a 3,500 MWCO SLIDE-A-LYZER cassette (Pierce). The full-length GP1 SEC eluate and dialyzed GP1 fragment pools were then combined and concentrated using an AMICON stirred cell unit fitted with a 3,000 NMWL ultrafiltration membrane (Millipore) at 55 psig nitrogen. The sample was further concentrated with a CENTRIPLUS YM-3 unit (Millipore) at 2,500 g at RT, then stored overnight at 4° C. Precipitated SDS was removed from the concentrated sample by centrifugation at 2,500 g at 0° C. The Purified LASV GP1 was immediately sterile-filtered using a 0.2-μm MILLEX GV syringe filter (Millipore), aliquoted, and stored at −20° C.

A 3-L shaker flask culture of pMAL-c2x:GP2-transformed Rosetta 2(DE3) cells was grown at 30° C. to an A₆₀₀=0.5-0.6 in cLB and then induced with a final IPTG concentration of 0.15 mM. After incubation for 3.5 h at 30° C., the cells were harvested by centrifugation for 10 min at ˜13,000 g. The resulting cell paste was frozen at −20° C., then thawed and resuspended in nine volumes of lysis buffer (20 mM TrisHCl, 200 mM NaCl, 10 mM EDTA, 1 mM DTT, pH 8.0). As described above, bacterial protease inhibitor cocktail and lysozyme were added to the suspension, and the reaction incubated at 37° C. with agitation. After 30 min, 1/10 volume of 1M MgSO₄ and 50 μL 2000 U/mL DNase I (Roche) per gram wet cell paste were added. The solution was incubated for an additional 30 min at 37° C. and then centrifuged at 15,000 g for 15 min at 4° C. The supernatant was further clarified by 0.2-μm filtration, then diluted two-fold with lysis buffer and applied to a 1.6×11 cm amylose column at 75 cm/h. The column was washed with five column volumes of equilibration buffer (20 mM TrisHCl, 200 mM NaCl, 1 mM EDTA, 1 mM DTT, pH 7.4), and the fusion protein eluted with equilibration buffer containing 10 mM maltose. SDS was added to a final concentration of 0.03% (w/v), followed by 10 μL of 1 mg/mL of Factor Xa per A₂₈₀=1 of amylose column eluate. The reaction mixture was then incubated for 17 h at 4° C. The solution was then concentrated three-fold using an AMICON stirred cell unit fitted with a 3,000 NMWL ultrafiltration membrane (Millipore) at 30 psig nitrogen. Subsequently, the solution was loaded onto a 2.6 cm×70 cm SUPERDEX 200 Prep Grade size exclusion column in ˜6 mL aliquots and then eluted with 30 mM MES, 154 mM NaCl, 0.1% SDS, pH 6.7, at 30 cm/h. GP2-containing fractions were pooled and concentrated, as described for GP1 purification. The sample was further concentrated with a CENTRIPLUS YM-3 unit (Millipore) at 2,500 g at RT and stored overnight at 4° C. Precipitated SDS was removed by centrifugation at 2,500 g at 0° C. Purified LASV GP2 was immediately sterile-filtered using a 0.2-μm MILLEX GV syringe filter (Millipore), aliquoted, and stored at −20° C.

### Example 6

### Expression and Purification of Recombinant LASV Proteins Transiently Expressed in Mammalian Cells

Recombinant LASV protein expression was analyzed in HEK-293T/17 cells transiently-transfected with mammalian expression vectors, which were prepared using the PURELINK HIPURE PLASMID FILTER MIDIPREP kit (Invitrogen). Briefly, 1×10⁶ cells were seeded per well of a poly-D-lysine-coated 6-well plate in 2 mL of complete Dulbecco's modified Eagle's medium (cDMEM). After overnight incubation at 37° C. with 5% CO₂, cells were transfected with unrestricted recombinant plasmid DNAs using the cationic lipid reagent LIPOFECTAMINE2000 (Invitrogen), according to the manufacturer's instructions. Transfections were incubated for 72 h at 37° C. with 5% CO₂, and subsequently, cell culture supernatants were collected and clarified by centrifugation. To prepare cell extracts from transfected cultures, cell monolayers were carefully washed twice with Ca⁺⁺- and Mg⁺⁺-free PBS, pH 7.4, and lysed in the wells with a mammalian cell lysis buffer comprised of 50 mM Tris buffer, pH 7.5, 1 mM EDTA, 0.1% SDS, 0.5% deoxycholic acid, 1% IGEPAL CA-360, and a protease inhibitor cocktail (Sigma), according to the manufacturer's instructions. The protein concentration in each cell extract was determined with a Bradford assay kit, as indicated by the manufacturer (Pierce). Approximately 10 μg of each extract was examined by SDS-PAGE in 10% or 12% NUPAGE NOVEX Bis-Tris gels, according to the manufacturer's specifications (Novex, San Diego, Calif.). Proteins were subsequently transferred to nitrocellulose membranes for western blot analysis, as described below.

To purify sGP1-FLAG protein, 110 mL of supernatant harvested from 293T/17 cells transiently transfected with construct sGP1-FLAG construct was clarified by centrifugation and 0.2-μm filtration. The clarified supernatant was loaded onto a 1.6×2.2 cm anti-FLAG M2 agarose column (Sigma) at 1 ml/min. The column was washed with 20 column volumes of equilibration buffer (20 mM TrisHCl, 154 mM NaCl, pH 7.4) and sGP1-FLAG was eluted with 100 μg/ml FLAG peptide (Sigma) in equilibration buffer. The fractions were analyzed by SDS-PAGE and western blot, and the sGP1-FLAG-containing fractions were pooled. The sGP1-FLAG eluate pool was concentrated ˜8-fold to a ˜2 mL final volume using a CENTRIPLUS YM-10 concentrator (Millipore) and then dialyzed against one-thousand volumes of 1×PBS, pH 7.4 using a 7K MWCO (molecular weight cut-off) SLIDE-A-LYZER cassette (Pierce). Following dialysis, the sample was concentrated as before to ˜0.9 mL, aliquoted, and stored at −20° C.

### Example 7

### Generation of Stable NS0 and CHO Cell Lines Expressing Recombinant LASV Proteins

Stable NS0 cell lines were generated by electroporating 1×10⁷ cells with 50 μg of Pvu I-linearized expression vector DNA using a single pulse of 250 V, 400 μFd, ˜6 msec time constant. Cells were immediately washed in complete RPMI 1640 media and pelleted by centrifugation. The cell pellet was resuspended in cRPMI supplemented with 600 μg/mL of Zeocin (antibiotic for clone selection) and then incubated at 37° C. with 5% CO₂ for 2-3 weeks in a T-75 cell culture flask to allow for selection and growth of stable cell lines. When the culture reached approximately 25% confluency, cells were harvested, washed in fresh selection medium, and cloned by limiting dilution cell cloning (LDCC). The remaining culture was expanded to confluency in a T-225 cell culture flask, then cryopreserved. Emerging clones from the LDCC steps were transferred to 24-well plates. When clones grew to confluency, most of the cells were harvested, then lysed, and protein extracts were prepared for SDS-PAGE and western blot analysis. The remaining cells were fed with fresh selection medium and returned to the incubator. Clones stably expressing LASV proteins were expanded and cryopreserved.

Stable CHO DG44 cells lines were generated by LIPOFECTAMINE2000-mediated transfection of 5×10⁶ cells seeded in 10-cm cell culture dishes, as per the manufacturer's instructions (Invitrogen), using 18 μg of Pvu I-linearized expression vector DNA and 1.8 μg of circular pTK-neo plasmid DNA (Novagen). CHO DG44 cells are mutant for the expression of functional dihydrofolate reductase (dhfr). At 48 h post-transfection, cells were gently harvested from the 10-cm culture dish by trypsinization, then diluted in 500 mL cMEM containing 50 nM methatrexate (MTX) and 500 μg/mL neomycin. Two hundred μL of diluted CHO cell suspension was plated per well of a flat bottom 96-well cell culture plate. Plates were incubated at 37° C. with 5% CO₂ and 90% relative humidity (Rh) for 2-3 weeks, until emerging colonies were greater than 75% confluent. Supernatants were assayed for the presence of secreted sGP1 proteins by enzyme-linked immunosorbent assay (ELISA), as described below. The highest producing stable CHO DG44 clones were expanded for further analysis, large-scale generation, and purification of recombinant proteins as well as for cryopreservation.

### Example 8

### Western Blot Analysis of Recombinant LASV Proteins

The identity of LASV proteins generated in bacterial and mammalian systems was confirmed by western blot analysis using a mix of six LASV-specific mAbs, described above, at a 1:1000 dilution. Preliminary work indicated that the LASV mAb mix was well suited for detection of native and denatured LASV proteins by ELISA and western blot, respectively (data not shown). For western blot analysis, proteins were transferred to 0.45-μM nitrocellulose membranes using XCELL II BLOT MODULES, according to the manufacturer's instructions (Invitrogen). Blocking and probing of membranes were performed in 1×PBS, pH 7.4, 5% non-fat dry milk (NFDM), 0.05% TWEEN-20, and 0.1% thymerosal. Washes were performed with 1×PBS, pH 7.4, 0.1% TWEEN-20 (wash buffer). Detection was performed with horse radish peroxidase (HRP)-conjugated secondary antibodies and tetramethylbenzidine (TMB) membrane substrate. Reactions were stopped by immersing developed membranes in water, followed by immediate high resolution scanning for permanent recording. When applicable, blots were stripped in 62.5 mM Tris-HCl, pH 6.7, 5 mM EDTA, 2% SDS, 100 mM β-mercaptoethanol, for 1 h at 50° C. in a sealed plastic bag with shaking Stripped membranes were subsequently washed extensively in wash buffer, then blocked and reprobed, as described above.

### Example 9

### Enzyme-Linked Immunosorbant Assay

ELISA was performed to detect sGP1 in supernatants of stable CHO DG44 mammalian cell cultures. Briefly, supernatants were diluted two-fold in 1×PBS, pH 7.4, and used to coat wells of a NUNC POLYSORP ELISA plate (Nunc, Denmark). Plates were subsequently blocked in 1×PBS, pH 7.4, 5% NFDM, 0.05% TWEEN-20, 0.1% thymerosal and then probed with anti-LASV GP1-specific mAb L52-74-7A in the same buffer. An HRP-conjugated goat anti-mouse IgG (H+L) polyclonal antibody reagent (KPL) and 3,3′,5,5′-tetramethylbenzidine (TMB) substrate (KPL) were used for ELISA development. Reactions were stopped with 0.5 M H₂SO₄ and were read at 450 nm in a MOLECULAR DYNAMICS THERMOMAX spectrophotometer, using SOFTMAX PRO analysis software.

To evaluate the potential use of bacterially expressed GP1, GP2, and NP proteins for diagnostic assays, we performed IgG and IgM capture ELISAs. For the IgG ELISA, high-affinity COSTAR 3590 96-well plates were coated with recombinant GP2 and NP (respectively) at a final concentration of 0.2 μg per well in PBS, pH 7.5. Plates were incubated overnight at 4° C., and washed three times with PBS-TWEEN 20 (PBST). Plates were then blocked for 90 min with 200 μL, of blocking solution consisting of 5% milk in PBST. Human LASV-specific convalescent serum was then added to each well in four-fold serial dilutions, beginning with a 1:100 dilution in blocking buffer. The plates were incubated for 1 h at 37° C. and washed three times with PBST. An Fc-specific human anti-IgG HRP-conjugated antibody (Bethyl Laboratories, Montgomery, Tex.) was then added to each well at a final dilution of 1:1500 in blocking buffer. After 1 h incubation, 100 μL of TMB substrate (KPL) was added to each well for 5 min, and the reaction was stopped by adding 100 μL of TMB stop solution (KPL).

### Example 10

### Plan for Invention Production

LASV, Junin virus (JUNV) and several other members of the Arenaviridae induce severe, often fatal hemorrhagic fevers, and are classified as Biosafety Level 4 and NIAID Biodefense Category A agents. In addition to high case fatality rates, arenaviruses have many features that enhance their potential as bioweapons. Arenaviruses have relatively stable virions, do not require passage via insect vectors, are transmitted easily by human-to-human contact and can be spread by simple means of dispersal. The ease of travel to and from endemic areas also permits easy access to LASV and other arenaviruses for use as bioweapons. A cluster of hemorrhagic fever cases in the United States caused by any arenavirus would be a major public health incident. The potential use of arenaviruses as biological weapons directed against civilian or military targets necessitates development of effective commercial diagnostics. The goal of our proposed project is to develop and validate multiagent diagnostic immunoassays for arenaviruses using recombinant antigens. These assays would be used to determine the attack agent following a deliberate release, and allow the virus used to be distinguished from other hemorrhagic fever viruses, such as dengue virus or Ebola virus, that may have similar case presentations. Development of rapid immunodiagnostic assays will also improve treatment of arenaviral diseases, facilitate studies to understand their prevalence and natural history, and ultimately lead to vaccines for preventing these major causes of morbidity and mortality.

Production Phases

Phase 1: Production and characterization of LASV antigens and monoclonal antibodies and development of prototype LASV antigen-capture and IgM and IgG antibody-capture assay:

1.1. LASV glycoproteins GPC, GP1 and GP2, and the nucleoprotein (NP) would be expressed in eukaryotic cell lines and/or bacteria (above examples).

1.2. The recombinant LASV proteins would be used to immunize mice. Hybridoma cells producing mAb that bind GPC, GP1, GP2 or NP would be cloned, selected and established as lines.

1.3. Recombinant LASV antigen-capture and immunoglobulin M (IgM) and immunoglobulin G (IgG) antibody-capture enzyme-linked immunosorbent assays (ELISA) would be developed. The ELISA would be directly compared for sensitivity and specificity to current assays based on BSL4 grown virus.

1.4. Production of selected LASV antigens and LASV-specific mAb would be scaled-up.

1.5. Confirmed LASV clinical samples and control samples would be field-collected from Guinea and Sierra Leone, and South America. Approximately 200 LASV-positive samples per year from the Lassa ward at Kenema General Hospital and two sites in Guinea are anticipated.

Phase 2: Development of optimized LASV antigen-capture and IgM and IgG antibody-capture ELISA, production of pilot lots of these assays, validation of assays using non-human primate and field-collected samples from humans, and direct comparison of the newly derived ELISA with RT-PCR based assays:

2.1. Recombinant LASV antigen-capture and IgM- and IgG-capture ELISA would be optimized for the ability to detect various strains of LASV, including Josiah, Macenta, Z132, LP and clinical isolates from diverse regions in the Lassa endemic range including Nigeria. ELISA assays under development would also be directly compared for sensitivity and specificity with immunofluorescence assay (IFA), virus culture and PCR detection.

2.2. Pilot lots of recombinant LASV Ag-capture and IgM- and IgG-capture ELISA would be produced.

2.3. The humoral immune response to LASV virus proteins would be evaluated in adult rhesus macaques, as validation of the antigen-capture and antibody-capture ELISA. Cross-reactive epitopes of arenavirus GP1, GP2 and NP based on murine MAb will be identified, including early IgM-specific epitopes that appear to be the most important diagnostically.

2.4. LASV ELISA would be tested at field stations established in Kenema, Sierra Leone and N′Zerekore, Guinea.

Phase 3: Development of optimized multiagent arenavirus antigen-capture and IgM and IgG antibody-capture ELISA, production of pilot lots of these assays, validation of assays using non-human primate and field-collected samples from humans, and direct comparison of the newly derived ELISA with RT-PCR based assays:

3.1. JUNV GPC, GP1, GP2, and NP would be expressed in eukaryotic cell lines and/or bacteria.

3.2. The recombinant JUNV proteins would be used to immunize mice. Hybridoma cells producing mAb that bind JUNV GPC, GP1, GP2 or NP would be cloned, selected and established as lines.

3.3. Recombinant JUNV antigen-capture and immunoglobulin M (IgM) and immunoglobulin G (IgG) antibody-capture enzyme-linked immunosorbent assays (ELISA) would be developed. The specificity of the antigen-capture and antibody-capture LASV ELISA would thus be expanded to include both Old World arenaviruses and New World arenaviruses that could potentially be used as bioweapons. The ELISA would be directly compared for sensitivity and specificity to current assays based on BSL4 grown virus.

3.4. Production of selected JUNV antigens and JUNV mAb plus other needed New World arenavirus antigens and mAb would be scaled-up and ELISA would be further optimized.

3.5. Pilot lots of recombinant antigen multiagent Ag-capture and IgM- and IgG-capture ELISA would be produced.

3.6. Confirmed serum samples from patients infected with New World arenaviruses would be obtained and field-tested on the newly developed multiagent ELISA.

Studies and Results

TABLE 5

| Phase | Task | Notes |
|---|---|---|
| 1.1 | Expression of LASV GPC, | Optimal expression for GP2 and NP obtained |
| | GP1 and GP2, and NP in | with pMALp2x expression system plasmid in |
| | eukaryotic cell lines and/or | the Rosetta strain of E. coli (refer to above |
| | bacteria | Examples). |
| 1.2 | Immunization of mice with | Mice immunized with recombinant LASV GP2 |
| | recombinant LASV proteins | or NP produced in E. coli. Mammalian GP1 and |
| | and cloning, selection and | GP2 immunizations completed. Archival clones |
| | establishment of hybridoma | of murine mAb specific for LASV GP1, GP2 |
| | lines. | and NP expanded. |
| 1.3 | Development of recombinant | Prototype assays developed and compared |
| | LASV antigen. IgG and IgM- | favorably for sensitivity and specificity to |
| | (ELISA) and direct | assays produced with BSL-4 grown LASV. |
| | comparison to current assays | |
| | based on BSL4 grown virus. | |
| 1.4 | Scale-up production of selected | All LASV antigens and mAbs produced to |
| | LASV antigens and LASV- | moderate scale sufficient for further assay |
| | specific mAb. | development. |
| 1.5 | Confirmed Lassa clinical | Human subjects protocols approved, field |
| | samples and control samples | stations established and sample collection |
| | will be field-collected from | begun. |
| | Guinea and Sierra Leone, and | |
| | South America. | |

TABLE 6

| Phase | Task | Notes |
|---|---|---|
| 2.1 | Optimize recombinant LASV | Recombinant ELISA assays have been directly |
| | Ag-capture and IgM- and | compared for sensitivity and specificity with |
| | IgG-capture ELISA to detect | non-recombinant assays and three variations of |
| | various strains of LASV | PCR detection. |
| 2.2 | Pilot lots of recombinant | Pilot lots of robust recombinant IgM and IgG |
| | LASV Ag-capture and IgM- | capture ELISA have been developed and |
| | and IgG-capture ELISA | produced. A robust antigen capture assay based |
| | have produced. | on a detection serum from animals immunized |
| | | to recombinant LASV proteins was also |
| | | produced. Configurations with Ag-capture mAb |
| | | based on mice immunized with recombinant |
| | | proteins would follow. |
| 2.3 | Identify cross-reactive | Epitope mapping (PEPSCAN) of approximately |
| | epitopes of arenavirus GP1, | 130 mAb produced to recombinant LASV |
| | GP2 and NP. | proteins would be performed. |
| 2.4 | Conduct field testing of | Prototype assays out-performed assays |
| | recombinant ELISA in West | produced with BSL-4 grown LASV and PCR in |
| | Africa. | field testing. |

Details Regarding Phase 2.1

LASV Ag-capture and IgM- and IgG-capture ELISA using recombinant LASV proteins and sera or monoclonal antibodies produced to these recombinant proteins have been optimized for sensitivity and specificity. Because RT-qPCR-based assays were able to be established in Sierra Leone, it was possible to compare the new ELISA assays to PCR-based assays in the field, which were considered to be a more stringent and appropriate test than the originally proposed IFA and virus culture comparison (see results below for phase 2.4). This also avoided the necessity of shipping samples potentially containing live LASV to the United States for PCR and confirmatory virus culture and was therefore less of a biosafety and biosecurity risk. The insensitive IFA test was not performed.

TABLE 7

| Traditional IgM capture ELISA | | | Recombinant IgM capture ELISA¹ | | |
|---|---|---|---|---|---|
| Step | Component | Time | Step | Component | Time |
| 1² | Anti-human IgM | 1 hour | — | | |
| 2² | Patient serum | 1 hour | 1³ | Patient serum | 30 min. |
| 3² | LASV- or mock- | 1 hour | — | | |
| | infected cell lysates | | | | |
| 4² | Rabbit anti-LASV | 1 hour | — | | |
| 5² | HRP-conj. anti-rabbit | 1 hour | 2³ | HRP-conj. anti-human | 30 min. |
| | IgG | | | IgM | |
| 6 | Tetramethylbenzidine | 5-15 min. | 3 | Tetramethylbenzidine | 5-15 min. |
| | (TMB); 2N H₂SO₄ | | | (TMB); 2N H₂SO₄ | |
| | (stop); read absorbance | | | (stop); read absorbance | |
| | at 450 nm | | | at 450 nm | |

¹Plates are pre-coated with recombinant LASV NP, GP1 and GP2.
²Plates are washed 5X after this step in PBS-TWEEN 20.
³Plates are washed 4X after this step in PBS-TWEEN 20.

A recombinant IgG capture ELISA has also been developed. It is identical to the recombinant IgM capture ELISA, except that an HRP-conjugated anti-human IgG antibody is used in step 2 of Table 7. Also, the traditional IgM capture assay was re-established in Sierra Leone to enable comparison to the recombinant IgM capture ELISA. This assay (Table 7) was similar to assays previously employed by CDC and USAMRIID except that rabbit anti-recombinant LASV protein serum was used. Attempts at reconstituting these traditional IgM capture assays were unsuccessful until the recombinant rabbit serum was substituted into the assays. These problems are believed to be due to lack of specificity of available sera prepared after injection of rabbits with disrupted cell culture-grown LASV.

An Ag-capture ELISA has also been produced that is based on a detection serum from animals immunized with recombinant LASV proteins. In this assay, murine mAbs to LASV proteins are coated onto ELISA plate wells. A 1:10 dilution of patient sera is added to the wells. Then, the detection serum from rabbits immunized with recombinant LASV proteins is added to detect the presence of the LASV antigens in the patient sera. Pilot lots of this Ag-capture ELISA were field-tested as described under phase 4. This assay was developed using an existing, but limited, set of mAb. These mAbs were produced in mice immunized with disrupted LASV produced in the BSL4.

Details Regarding Phase 2.2

Pilot lots of recombinant IgM and IgG capture ELISA have been produced. Also produced are pilot lots of the Ag-capture ELISA based on a detection serum from animals immunized with recombinant LASV proteins.

Details Regarding Phase 2.3

The humoral immune response to LASV virus proteins would be evaluated in adult rhesus macaques, as a means to validate the antigen-capture and antibody-capture ELISA. Cross-reactive epitopes of arenavirus GP1, GP2 and NP based on murine mAb will be identified, including early IgM-specific epitopes that appear to be the most important diagnostically. Serum from guinea pigs and rhesus macaques challenged with LASV would be tested in recombinant antigen LASV Ag-capture and IgM- and IgG-capture ELISA during the remainder of the grant period, but do not consider this testing to be essential for completion of the project.

Over 130 individual clones of cells producing mAbs to recombinant LASV NP, GP1 or GP2 have been produced. The mAbs react to the proteins to which the mice were immunized in ELISA-based assays and in some cases western blotting. These mAbs would be tested for reactivity in PEPSCAN assays in which mAbs are tested for binding to overlapping peptides designed from LASV NP, GP1 and GP2. This epitope mapping would allow the selection of mAbs with non-overlapping specificities for incorporation into the Ag-capture ELISA as described above.

TABLE 8¹

| | | | Trad. | | Rec | | Ag- | Ag- | Ag- |
|---|---|---|---|---|---|---|---|---|---|
| | | Ag- | IgM | Rec. | IgM + | PCR + | capture | capture | capture + |
| | | capture | (tIgM) | IgM | rIgG | tIgM | plus | plus | rIgM/ |
| | PCR² | ELISA* | ELISA | ELISA | ELISA | ELISA | tIgM | rIgM | rIgG |
| True | 13 | 19 | 11 | 24 | 26 | 20 | 22 | 25 | 26 |
| Positive | | | | | | | | | |
| True | 11 | 11 | 11 | 12 | 12 | 10 | 11 | 12 | 12 |
| Negative | | | | | | | | | |
| False | 1 | 0 | 1 | 0 | 0 | 2 | 1 | 0 | 0 |
| Positive | | | | | | | | | |
| False | 13 | 8 | 15 | 2 | 0 | 6 | 4 | 1 | 0 |
| Negative | | | | | | | | | |
| Sensitivity³ | 50.0% | 73.1% | 42.3% | 92.3% | 100% | 76.9% | 84.6% | 96.2% | 100% |
| Specificity⁴ | 91.7% | 100% | 91.7% | 100% | 100% | 83.3% | 91.7% | 100% | 100% |
| Efficiency⁵ | 68.4% | 78.9% | 57.9% | 94.7% | 100% | 78.9% | 86.8% | 97.4% | 100% |

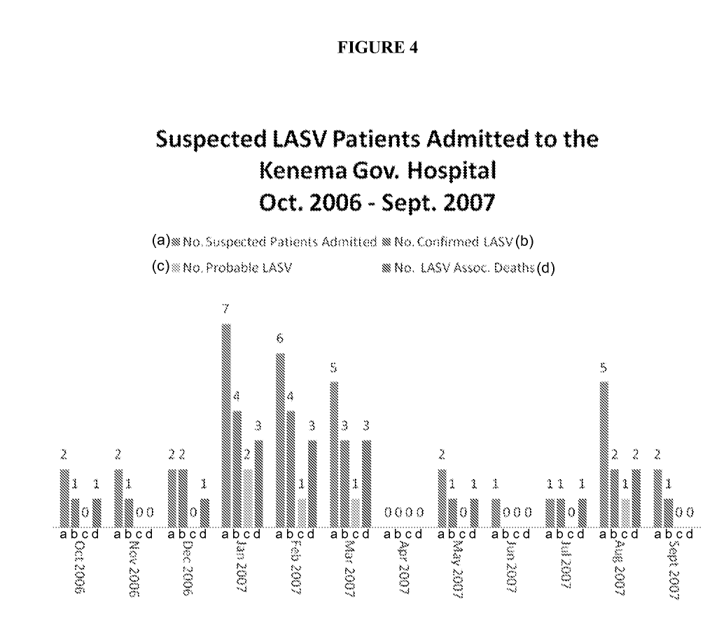

¹96 serum samples from 38 consecutive well-characterized patents attending the KGH Lassa Ward were analyzed by PCR and various ELISA. 26 patients (True Positives; TP) were judged to have acute Lassa infection (most were the “confirmed” or “probable” cases from FIG. 4). 12 patients (True Negatives; TN) were judged to have either non-Lassa febrile illness (n = 6) or to have had past Lassa viruses infection (n = 6; 4 of these were follow-up patients from the Lassa ward).
²samples were scored positive if positive on one or more of three real-time RT-qPCR assays: USAMRIID standard PCR, WHO alternative PCR or pan-arenavirus PCR.
³TP/TP + FN × 100.
⁴TN/TN + FP × 100.
⁵TP + TN/Total × 100.

Details Regarding Phase 2.4

The recombinant ELISAs under development have been directly compared for sensitivity and specificity with non-recombinant ELISAs and three variations of PCR detection in the diagnostics laboratory at Kenema Government Hospital (KGH). This field testing initially focused on samples from a serological panel consisting of 96 serum samples from 38 consecutive well-characterized patients attending the KGH Lassa Ward (FIG. 4). Current WHO (World Health Organization) guidelines consider that a patient can be considered a “confirmed” case only if the Ag-capture assay is positive, but can be considered a “suspected” case, if the IgM assay is positive. It is expected that the recombinant ELISAs under development would permit a revision of these guidelines. Thus, for the purposes of the current analysis 26 of the 38 patients were considered to be True Positives for acute LASV infection based on assay results, clinical signs and symptoms. Twelve patients in the serological panel were true negatives for acute LASV infection based on the same criteria. Six of these sign- and symptom-free patients were judged to have had past LASV infections based on negative PCR and Ag-capture, but positive IgM or IgG serology.

Comparison of these assays revealed that the recombinant antigen based assays out-performed the PCR assays and traditional ELISA, by measures of sensitivity, specificity and efficiency (Table 8). Sensitivity for three PCR assays combined was 50% compared to 73% for the Ag-capture assay. Individually, none of the three PCR assays approached 50% sensitivity. The most sensitive was the USAMRIID standard PCR with 31% sensitivity (92% specificity). As described above, this version of the Ag-capture ELISA uses sera from rabbits immunized with recombinant LASV proteins as a detection reagent, and is more sensitive and specific than prior versions of this ELISA. Both PCR and Ag-capture ELISA require the presence of virions, viral RNA or viral proteins in the patient sample. LASV viremia is known to be transient, with virus from peripheral blood cleared rapidly by both innate and early acquired immune responses. The True Positive patients in this cohort had a case mortality of 83%, which is higher than expected. Generally, patients coming to the KGH ward (which has only recently been able to perform any LASV testing) had more severe Lassa than in other past surveys. It is suspected that they were also further along in the disease course.

The recombinant IgM assay was more than twice as sensitive (42 vs. 92%) and more specific (92 vs. 100%) than the traditional (Centers for Disease Control; CDC) IgM assay (Table 8, see also Table 5). When combined with the rIgG capture assay, IgM capture detected 100% of the True Positive acute Lassa patients, with no false positives. While these results are intriguing, it is important to keep in mind that this cohort of patients may be further along in the disease course than in other past surveys. Patients presenting early in the disease course, while viremic, but before the development of an antibody response, would be expected to be negative even based on the above ultrasensitive IgG and IgM capture ELISA. Therefore, further development of the Ag-capture ELISA would be essential to detect such early Lassa patients, prior to the antibody response to the virus. It should also be noted that a subset of the acute Lassa patients (2 out of 26) did not present with a detectable IgM response, but rather an IgG only response. This type of response has been noted before (Bausch et al, 2000). The practical aspect of this observation is that IgG capture would be used in combination with IgM capture for acute diagnosis of Lassa in addition to being an important assay for LASV surveillance.

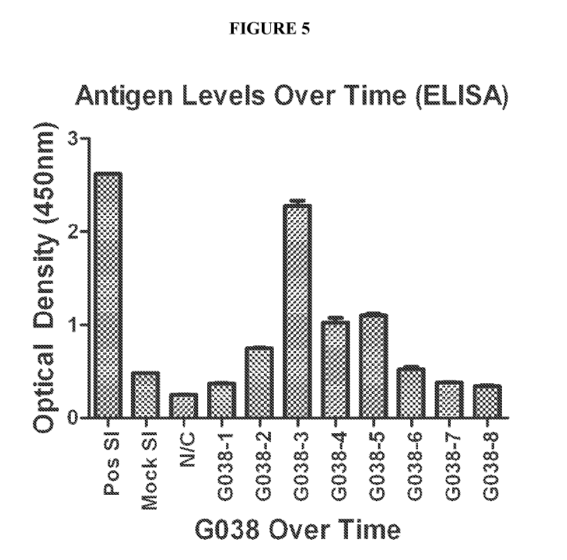

The analyses in Table 8 are generally based in testing of two or more serum samples per patient. An example of LASV Ag levels in one patient with several available samples is shown in FIG. 5. As expected, antibody responses typically followed PCR or antigen positivity. There were, however, many patients in which LASV RNA (PCR) or Ag and LASV-specific IgM or IgG were detected in the same sample, which has been observed only rarely in past surveys with traditional (CDC) assays. This situation reflects the increased sensitivity of the newly developed recombinant assays.

Prior studies indicated that combining the Ag-capture ELISA with the IgM capture ELISA was the most sensitive and specific method for diagnosing acute Lassa. Indeed, the above results combining the Ag-capture ELISA with the traditional IgM capture give sensitivities and specificities that were very similar to those of previous surveys (Table 8) (Bausch et al., 2000). PCR did not perform as well in combination with the traditional IgM (tIgM) capture. Therefore, it cannot be recommended that the current USAMRIID PCR or any combination of available PCRs be used as a substitute for the Ag-capture assay for diagnosing patients in the early stages of Lassa.

These results suggest that combining a recombinant Ag-capture with a recombinant IgG/IgM capture assay would provide the most sensitive and specific method for diagnosing acute Lassa. Thus, conversion of these ELISAs to lateral flow assays (similar to pregnancy tests) is envisioned to provide a point-of-care diagnostic that can be useful in many situations particularly in East African villages without access to ELISA equipment.

TABLE 9¹

| | | | | | | | One or more | |
|---|---|---|---|---|---|---|---|---|
| | rNucleoprotein | | rGlycoprotein 1 | | rGlycoprotein 2 | | rLASV proteins | |
| | Follow- | | Follow- | | Follow- | | Follow- | |
| | up | Contact | up | Contact | up | Contact | up | Contact |
| Positive | 13 | 1 | 7 | 5 | 9 | 6 | 15 | 11 |
| IgG | (59%) | (6%) | (32%) | (26%) | (41%) | (32%) | (68%) | (58%) |
| Negative | 9 | 18 | 15 | 14 | 13 | 13 | 7 | 8 |
| IgG | (41%) | (94%) | (68%) | (74%) | (59%) | (68%) | (32%) | (42%) |
| Positive | 3 | 0 | 7 | 3 | 7 | 2 | 11 | 5 |
| IgM | (17%) | (0%) | (32%) | (16%) | (32%) | (11%) | (50%) | (26%) |
| Negative | 19 | 19 | 15 | 16 | 15 | 17 | 11 | 14 |
| IgM | (86%) | (100%) | (68%) | (84%) | (68%) | (89%) | (50%) | (74%) |

¹22 patients who attended the KGH Lassa ward in either 2004 or 2005 were contacted and asked to give a blood sample. In addition, 19 household contacts, who did not develop clinical signs of Lassa, also agreed to give blood samples.

To further characterize the recombinant LASV ELISA, serum samples from 22 follow-up patients who attended the KGH Lassa ward in either 2004 or 2005 (Table 9) were tested. In addition, 19 household contacts who did not develop clinical signs of Lassa also agreed to give blood samples. As expected, a subset of patients retained both IgM and IgG LASV responses detectable by ultrasensitive ELISA. It is of interest that household contacts of Lassa patients had similar prevalence levels of LASV specific IgG and about a third to half as prevalent LASV specific IgM. It has been speculated that non-apparent Lassa can occur, i.e. that LASV exposure does not produce clinical Lassa in all exposed patients. Contacts of Lassa patients were at increased risk for LASV infection either from the environmental conditions that resulted in the patient infections or from the patient. Subclinical Lassa could produce the antibody responses shown here. Studies are in progress to determine the prevalence levels of LASV specific IgG/IgM in the general population, but it is likely to be less than observed in follow-up patients from the Lassa ward or their household contacts. It is of interest that follow-up patients were more likely to produce IgG specific for LASV NP compared to contacts (Table 9).

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the claims.

### REFERENCES

1. Auperin, D. D., Sasso, D. R. and McCormick, J. B. (1986). Nucleotide sequence of the glycoprotein gene and intergenic region of the Lassa virus S genome RNA. Virology 154, 155-167.
2. Beyer, W. R., Popplau, D., Garten, W., von Laer, and Lenz O. (2003). Endoproteolytic processing of the lymphocytic choriomeningitis virus glycoprotein by the sibtilase SKI-1/S1P. J. Virol. 77, 2866-2872.
3. Buchmeier, M. J. (2002). Arenaviruses: protein structure and function. Curr. Top. Microbiol. Immunol. 262, 259-173.
4. Buchmeier, M. J., and Parekh, B. S. (1987). Protein structure and expression among arenaviruses. Current Topics in Microbiology and Immunology 133, 41-57.
5. Buchmeier, M. J., Lewicki, H. A., Tomor, O., and Johnson, K. M. (1980). Monoclonal antibodies to lymphocytic choriomeningitis virus reacts with pathogenic arenaviruses. Nature, London 288, 4876-4877.
6. Burnette, W. N. (1981). “Western Blotting”: electrophoretic transfer of proteins from sodium dodecyl sulfate-polyacrylamide gels to unmodified nitrocellulose and radiographic detection with antibody and radioiodinated protein A. Analytical Biochemistry 112, 195-203.
7. Clegg, J. C. and Lloyd, G. (1983). Structureal and cell-associated proteins of Lassa virus. Journal of General Virology 64, 1127-1136.
8. Eichler, R., Lenz, O., Strecker, T., Eickmann, M., Klenk, H. D., and Garten, W. (2004). Lassa virus glycoprotein signal peptide displays a novel topology with an extended ER-luminal region. J. Biol. Chem. 279, 12293-12299.
9. Eichler, R., Lenz, O., Strecker, T., Eickmann, M., Klenk, H. D., and Garten, W. (2003). Identification of Lassa virus glycoprotein signal peptide as a trans-acting maturation factor. EMBO Rep. 4, 1084-1088.
10. Eichler, R., Lenz, O., Strecker, T., Eickmann, and Garten, W. (2003). Signal peptide of Lassa virus glycoprotein GP-C exhibits an unusual length. FEBS Lett. 538, 203-206.
11. Elagoz, A., Benjannet, S., Mammarbassi, A., Wickham, L., and Seidah, N. G. (2002). Biosynthesis and cellular trafficking of the convertase SKI-1/S1P: ectodomain shedding requires SKI-1 activity. J. Biol. Chem. 277, 11265-11275.
12. Hufert, F. T., Ludke, W., and Schmitz, H. (1989). Epitope mapping of the Lassa virus nucleocapsid protein using monoclonal anti-nucleocapsid antibodies. Archives of Virology 106, 201-212.
13. Lenz, O., ter Meulen, J., Feldmann, H., Lenk, H.-D., and Garten, W. (2000). Identification of a novel consensus sequence at the cleavage site of the Lassa virus glycoprotein. J. Virol. 74, 11418-11421.
14. Lukashevich L. S., Clegg J. C., and Sidibe K. (1993). Lassa virus activity in Guinea: distribution of human antiviral antibody defined using enzyme-linked immunosorbent assay with recombinant antigen. J Med Virol. 40, 210-7.
15. McCormick, J. B., and Fisher-Hoch, S. P. (2002). Lassa Fever. Curr. Top. Microbiol. Immunol. 262, 75-109.
16. Ruo, S. L., Mitchell, S. W., Killey, M. P., Roumillat, L. F., Fisher-Hoch, S. P., and McCormick, J. B. (1991). Antigenic relatedness beTWEEN arenaviruses defined at the epitope level by monoclonal antibodies. Journal of General Virology 72, 549-555.
17. Sanchez, A., Pifat, D. Y., Kenyon, R. H., Peters, C. J., McCormick, J. B., and Kiley, M. P. (1989). Junin virus monoclonal antibodies: characterization and cross-reactivity with other arenaviruses. J. Gen. Virol. 70, 1125-1132.
18. Spiropoulou, C. F., Kunz, S., Rollin, P. E., Campbell, K. P., and Oldstone, M. B. A. (2002). New World arenavirus Glade C, but not Glade A and B viruses, utilizes a-dystroglycan as its major receptor. J. Virol. 76, 5140-5146.
19. ter Meulen J., Badusche M., Kuhnt K., Doetze A., Satoguina J., Marti T., Loeliger C., Koulemou K., Koivogui L., Schmitz H., Fleischer B., and Hoerauf A. (2000). Characterization of human CD4(+) T-cell clones recognizing conserved and variable epitopes of the Lassa virus nucleoprotein. J. Virol. 74, 2186-92.
20. ter Meulen, J., Koulemou K., Wittekindt T., Windisch K., Strigl S., Conde S., and Schmitz H. (1998). Detection of Lassa Virus Antinucleoprotein Immunoglobulin G (IgG) and IgM Antibodies by a Simple Recombinant Immunoblot Assay for Field Use. J. Clin. Microbiol. 36, 3143-3148.
21. York, J., Agnihothram, S. S., Ronamowski, V., and Nunberg, J. H. (2005). Genetic analysis of heptad-repeat regions in the G2 fusion subunit of the Junin arenavirus envelope glycoprotein. Virology 343, 267-279.
22. York, J., Ronamowski, V., Lu, M., and Nunberg, J. H. (2004). The signal peptide of the Junin arenavirus envelope glycoprotein is myristoylated and forms an essential subunit of the mature G1-G2 complex. J. Virol. 78, 10783-10792.

1. A polypeptide comprising an isolated protein selected from the group consisting of (a) a polypeptide consisting of the amino acid sequence of the ectodomain of Lassa virus (LASV) glycoprotein 1 (GP1) optionally fused at the C-terminus thereof to an amino acid sequence consisting of the transmembrane domain of LASV glycoprotein 2 (GP2), (b) a polypeptide consisting of the ectodomain of LASV GP2, and (c) a full length, soluble recombinant Lassa virus nucleocapsid protein (LASV NP) fused with maltose binding protein (MBP).

2. (canceled)

3. The polypeptide of claim 1, wherein the isolated protein is reactive with antibodies from human LASV-specific convalescent serum.

4. (canceled)

5. The polypeptide of claim 1, wherein the isolated protein is expressed in Escherichia coli (E. coli) cells.

6. The polypeptide of claim 5, wherein the E. coli cells comprise E. coli Rosetta gami 2 cells.

7. An immunoassay kit for diagnosing a Lassa virus infection, the kit comprising the polypeptide of claim 1.

8. The immunoassay kit of claim 7, wherein the immunoassay is an enzyme-linked immunosorbant assay.

9. The immunoassay kit of claim 7, wherein the kit further comprises an antibody that is specific for Lassa virus nucleocapsid protein.

10-17. (canceled)

18. The immunoassay kit of claim 7, wherein the kit further comprises an antibody that is specific for Lassa virus GP1 protein.

19. The immunoassay kit of claim 7, wherein the kit further comprises an antibody that is specific for Lassa virus GP2 protein.

20. The polypeptide of claim 1, wherein the isolated peptide is fused at the N-terminus thereof to an amino acid sequence selected from the group, human λ light chain, human heavy chain signal peptide, and the Flag-tag sequence (DYKDDDDKG).

21. The polypeptide of claim 1, wherein the isolated peptide is the polypeptide consisting of the amino acid sequence of the ectodomain of Lassa virus (LASV) glycoprotein 1 (GP1) optionally fused at the C-terminus thereof to an amino acid sequence consisting of the transmembrane domain of LASV glycoprotein 2 (GP2).

22. The polypeptide of claim 21, wherein the isolated peptide is fused at the N-terminus thereof to an amino acid sequence selected from the group, human λ light chain, human heavy chain signal peptide, and the Flag-tag sequence (DYKDDDDKG).

23. The polypeptide of claim 1, wherein the isolated peptide is the polypeptide consisting of the amino acid sequence of the ectodomain of Lassa virus (LASV) glycoprotein 1 (GP1) fused at the C-terminus thereof to an amino acid sequence consisting of the transmembrane domain of LASV glycoprotein 2 (GP2).

24. The polypeptide of claim 23, wherein the isolated peptide is fused at the N-terminus thereof to an amino acid sequence selected from the group, human λ light chain, human heavy chain signal peptide, and the Flag-tag sequence (DYKDDDDKG).

25. The polypeptide of claim 1, wherein the isolated peptide is the polypeptide consisting of the ectodomain of LASV GP2.

26. The polypeptide of claim 25, wherein the isolated peptide is fused at the N-terminus thereof to an amino acid sequence selected from the group, human λ light chain, human heavy chain signal peptide, and the Flag-tag sequence (DYKDDDDKG).

27. An immunoassay kit for diagnosing a Lassa virus infection, the kit comprising the polypeptide of claim 21.

28. An immunoassay kit for diagnosing a Lassa virus infection, the kit comprising the polypeptide of claim 23.

29. An immunoassay kit for diagnosing a Lassa virus infection, the kit comprising the polypeptide of claim 25.

30. The polypeptide of claim 1, wherein the isolated peptide is the polypeptide consisting of the full length, soluble recombinant Lassa virus nucleocapsid protein (LASV NP) fused with maltose binding protein (MBP).


SEQUENCE LISTING (30 sequences)

SEQ ID NO: 1 - DNA, 57 bp, Artificial Sequence
  Synthetic
atccagggaaggatttcagaattcggatcctctagagtcgacctgcaggcaagcttg

SEQ ID NO: 2 - protein, 4 aa, Artificial Sequence
  Synthetic
IEGR

SEQ ID NO: 3 - protein, 8 aa, Artificial Sequence
  Synthetic
DYKDDDDK

SEQ ID NO: 4 - DNA, 1710 bp, Artificial Sequence
  Synthetic
atgagtgcctcaaaggaaataaaatcctttttgtggacacaatctttgaggagggaattatctggttactgctccaacatcaaactacaggtggtgaaagatgcccaggctcttttacatggacttgacttctccgaagtcagtaatgttcaacggttgatgcgcaaggagagaagggatgacaatgatttgaaacggttgagggacctaaatcaagcggtcaacaatcttgttgaattaaaatcaactcaacaaaagagtatactgagagttgggactctaacctcagatgacttattaatcttagccgctgatctagagaagttaaagtcaaaggtgatcagaacagaaaggccattaagtgcaggtgtctatatgggcaacctaagctcacagcaacttgaccaaagaagagctctcctgaatatgataggaatgagtggtggtaatcaaggggctcgggctgggagagatggagtggtgagagtttgggatgtgaaaaatgcagagttgctcaataatcagttcgggaccatgccaagtctgacactggcatgtctgacaaaacaggggcaggttgacttgaatgatgcagtacaagcattgacagatttgggtttgatctacacagcaaagtatcccaacacttcagacttagacaggctgactcaaagtcatcccatcctaaatatgattgacaccaagaaaagctctttgaatatctcaggttataattttagcttgggtgcagctgtgaaggcaggagcttgcatgctggatggtggcaatatgttggagacaatcaaggtgtcacctcagacaatggatggtatcctcaaatccattttaaaggtcaagaaggctcttggaatgttcatttcagacacccctggtgaaaggaatccttatgaaaacatactctacaagatttgtttgtcaggagatggatggccatatattgcatcaagaacctcaataacaggaagggcctgggaaaacactgtcgttgatctggaatcagatgggaagccacagaaagctgacagcaacaattccagtaaatccctgcagtcggcagggtttaccgctgggcttacctattctcagctgatgaccctcaaggatgcaatgctgcaacttgacccaaatgctaagacctggatggacattgaaggaagacctgaagatccagtggaaattgccctctatcaaccaagttcaggctgctacatacacttcttccgtgaacctactgatttaaagcagttcaagcaggatgctaagtactcacatgggattgatgtcacagacctcttcgctacacaaccgggcttgaccagtgctgtcattgatgcactcccccggaatatggtcattacctgtcaggggtccgatgacataaggaaactccttgaatcacaaggaagaaaagacattaaactaattgatattgccctcagcaaaactgattccaggaagtatgaaaatgcagtctgggaccagtataaagacttatgccacatgcacacaggtgtcgttgttgaaaagaagaaaagaggcggtaaagaggaaataacccctcactgtgcactaatggactgcatcatgtttgatgcagcagtgtcaggaggactgaacacatcggttttgagagcagtgctgcccagagatatggtgttcagaacatcgacacctagagtcgttctgtaa

SEQ ID NO: 5 - protein, 569 aa, Artificial Sequence
  Synthetic
MSASKEIKSFLWTQSLRRELSGYCSNIKLQVVKDAQALLHGLDFSEVSNVQRLMRKERRDDNDLKRLRDLNQAVNNLVELKSTQQKSILRVGTLTSDDLLILAADLEKLKSKVIRTERPLSAGVYMGNLSSQQLDQRRALLNMIGMSGGNQGARAGRDGVVRVWDVKNAELLNNQFGTMPSLTLACLTKQGQVDLNDAVQALTDLGLIYTAKYPNTSDLDRLTQSHPILNMIDTKKSSLNISGYNFSLGAAVKAGACMLDGGNMLETIKVSPQTMDGILKSILKVKKALGMFISDTPGERNPYENILYKICLSGDGWPYIASRTSITGRAWENTVVDLESDGKPQKADSNNSSKSLQSAGFTAGLTYSQLMTLKDAMLQLDPNAKTWMDIEGRPEDPVEIALYQPSSGCYIHFFREPTDLKQFKQDAKYSHGIDVTDLFATQPGLTSAVIDALPRNMVITCQGSDDIRKLLESQGRKDIKLIDIALSKTDSRKYENAVWDQYKDLCHMHTGVVVEKKKRGGKEEITPHCALMDCIMFDAAVSGGLNTSVLRAVLPRDMVFRTSTPRVVL

SEQ ID NO: 6 - DNA, 1479 bp, Artificial Sequence
  Synthetic
aaaatgggacaaatagtgacattcttccaggaagtgcctcatgtaatagaagaggtgatgaacattgttctcattgcactgtctgtactagcagtgctgaaaggtctgtacaattttgcaacgtgtggccttgttggtttggtcactttcctcctgttgtgtggtaggtcttgcacaaccagtctttataaaggggtttatgagcttcagactctggaactaaacatggagacactcaatatgaccatgcctctctcctgcacaaagaacaacagtcatcattatataatggtgggcaatgagacaggactagaactgaccttgaccaacacgagcattattaatcacaaattttgcaatctgtctgatgcccacaaaaagaacctctatgaccacgctcttatgagcataatctcaactttccacttgtccatccccaacttcaatcagtatgaggcaatgagctgcgattttaatgggggaaagattagtgtgcagtacaacctgagtcacagctatgctggggatgcagccaaccattgtggtactgttgcaaatggtgtgttacagacttttatgaggatggcttggggtgggagctacattgctcttgactcaggccgtggcaactgggactgtattatgactagttatcaatatctgataatccaaaatacaacctgggaagatcactgccaattctcgagaccatctcccatcggttatctcgggctcctctcacaaaggactagagatatttatattagtagaagattgctaggcacattcacatggacactgtcagattctgaaggtaaagacacaccagggggatattgtctgaccaggtggatgctaattgaggctgaactaaaatgcttcgggaacacagctgtggcaaaatgtaatgagaagcatgatgaggaattttgtgacatgctgaggctgtttgacttcaacaaacaagccattcaaaggttgaaagctgaagcacaaatgagcattcagttgatcaacaaagcagtaaatgctttgataaatgaccaacttataatgaagaaccatctacgggacatcatgggaattccatactgtaattacagcaagtattggtacctcaaccacacaactactgggagaacatcactgcccaaatgttggcttgtatcaaatggttcatacttgaacgagacccacttttctgatgatattgaacaacaagctgacaatatgatcactgagatgttacagaaggagtatatggagaggcaggggaagacaccattgggtctagttgacctctttgtgttcagcacaagtttctatcttattagcatcttccttcacctagtcaaaataccaactcataggcatattgtaggcaagtcgtgtcccaaacctcacagattgaatcatatgggcatttgttcctgtggactctacaaacagcctggtgtgcctgtgaaatggaagagatga

SEQ ID NO: 7 - protein, 491 aa, Artificial Sequence
  Synthetic
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 8 - DNA, 60 bp, Artificial Sequence
  Synthetic
atggcctggtctcctctcctcctcactctcctcgctcactgcacagggtcctgggcccag

SEQ ID NO: 9 - protein, 20 aa, Artificial Sequence
  Synthetic
MAWSPLLLTLLAHCTGSWAQ

SEQ ID NO: 10 - DNA, 57 bp, Artificial Sequence
  Synthetic
atgggctggagctgcatcatcctgttcctggtggccaccgccaccggcgtgcacagc

SEQ ID NO: 11 - protein, 19 aa, Artificial Sequence
  Synthetic
MGWSCIILFLVATATGVHS

SEQ ID NO: 12 - DNA, 41 bp, Artificial Sequence
  Synthetic
tttcagaattcggatccaccagtctttataaaggggtttat

SEQ ID NO: 13 - DNA, 49 bp, Artificial Sequence
  Synthetic
ggtaccaagctttcagtcatagcaatcttctactaatataaatatctct

SEQ ID NO: 14 - DNA, 38 bp, Artificial Sequence
  Synthetic
tttcagaattcggatccggcacattcacatggacactg

SEQ ID NO: 15 - DNA, 40 bp, Artificial Sequence
  Synthetic
ggtaccaagctttcagctatgtcttcccctgcctctccat

SEQ ID NO: 16 - DNA, 77 bp, Artificial Sequence
  Synthetic
tttcagaattcagtgcctcaaaggaaataaaatcctttttgtggacacaatctttgaggagggaattatctggttac

SEQ ID NO: 17 - DNA, 43 bp, Artificial Sequence
  Synthetic
ggtaccaagctttcagttacagaacgactctaggtgtcgatgt

SEQ ID NO: 18 - DNA, 36 bp, Artificial Sequence
  Synthetic
gtagctagcatgggacaaatagtgacattcttccag

SEQ ID NO: 19 - DNA, 40 bp, Artificial Sequence
  Synthetic
ggtaccaagctttcagtcatctcttccatttcacaggcac

SEQ ID NO: 20 - DNA, 130 bp, Artificial Sequence
  Synthetic
ggtaccaagctttcagtcatggtattttgactaggtgaaggaagatgctaataagatagaaacttgtgctgaacacaaagaggtcaactagacccaatggtagcaatcttctactaatataaatatctct

SEQ ID NO: 21 - DNA, 65 bp, Artificial Sequence
  Synthetic
cgataagctttcagtcagcccttgtcgtcgtcgtccttgtagtctagcaatcttctactaatata

SEQ ID NO: 22 - DNA, 97 bp, Artificial Sequence
  Synthetic
gatcgctagcgccgccaccatgggctggagctgcatcatcctgttcctggtggccaccgccaccggcgtgcacagcaccagtctttataaaggggtt

SEQ ID NO: 23 - DNA, 97 bp, Artificial Sequence; identical to SEQ ID NO: 22
  Synthetic

SEQ ID NO: 24 - DNA, 97 bp, Artificial Sequence
  Synthetic
aagctggctagccaccatggcctggtctcctctcctcctcactctcctcgctcactgcacagggtcctgggcccagggcacattcacatggacactg

SEQ ID NO: 25 - DNA, 97 bp, Artificial Sequence
  Synthetic
gatcgctagcgccgccaccatgggctggagctgcatcatcctgttcctggtggccaccgccaccggcgtgcacagcggcacattcacatggacactg

SEQ ID NO: 26 - DNA, 97 bp, Artificial Sequence; identical to SEQ ID NO: 24
  Synthetic

SEQ ID NO: 27 - DNA, 43 bp, Artificial Sequence
  Synthetic
ggtaccaagctttcagtcatggtattttgactaggtgaaggaa

SEQ ID NO: 28 - DNA, 97 bp, Artificial Sequence; identical to SEQ ID NO: 25
  Synthetic

SEQ ID NO: 29 - DNA, 65 bp, Artificial Sequence
  Synthetic
cgataagctttcagtcagcccttgtcgtcgtcgtccttgtagtctgtcttcccctgcctctccat

SEQ ID NO: 30 - protein, 9 aa, Artificial Sequence
  Synthetic
DYKDDDDKG